(19) United States
(12) Patent Application Publication
(10) Pub. No.: US 2026/0224463 A1
(43) Pub. Date: Aug. 6, 2026

(54) COMPOSITIONS COMPRISING HYDROPHILIC ACTIVE INGREDIENTS IN OIL

(71) Applicant: Lubrizol Advanced Materials, Inc., Cleveland, OH (US)

(72) Inventors: Nelia DELGADO COBOS, Gavá (ES); Laura Isabel GIRALDO URREA, Cunit (ES); Marine VINCENDET, Terrassa (ES); Cristina AMEZCUA FONTRODONA, Barcelona (ES)

(73) Assignee: Lubrizol Advanced Materials, Inc., Cleveland, OH (US)

(21) Appl. No.: 19/153,035

(22) PCT Filed: Feb. 1, 2024
(86) PCT No.: PCT/IB2024/050923

§ 371 Date: Aug. 1, 2025

(30) Foreign Application Priority Data
Feb. 2, 2023 (EP) 23382094.3

(51) Int. Cl. A61K 8/64; A61K 47/14; A61K 47/26; A61K 47/44; A61Q 19/00

(52) U.S. Cl. CPC A61K 8/64; A61K 47/14; A61K 47/26; A61K 47/44; A61Q 19/00

(57) ABSTRACT

A composition comprising: i) from 2 to 5 wt. % of an aqueous solution comprising a hydrophilic active ingredient; ii) from 1 to 2 wt. % of methyl glucose sesquistearate; iii) from 10 to 12 wt. % of a polyglyceryl-3 ester of a fatty acid; iv) at least 83 wt. % of an ester oil selected from the group consisting of cetyl ethylhexanoate, isopropyl isostearate and ethylhexyl palmitate; and wherein all the weight percentages are based on the total weight of the composition; and, wherein the sum of i), ii), iii) and iv) does not exceed 100 wt. %. A method for dispersing a hydrophilic active ingredient in oil. A cosmetic or dermopharmaceutical formulation which comprises said initial composition. A method of cosmetic, non-therapeutic treatment and/or care of the skin, hair, nails and/or mucous membranes of a subject comprising topically administering a cosmetically effective amount of said compositions.

This application claims the priority of International Application PCT/IB2024/050923, filed Feb. 1, 2024, and EP 23382094.3, filed Feb. 2, 2023, the disclosures of which are incorporated herein by reference in their entireties.

### FIELD OF THE INVENTION

The present invention relates to compositions comprising hydrophilic active ingredients in oil systems, more particularly hydrophilic cosmetic and dermopharmaceutical active ingredients. The compositions are useful for the preparation of cosmetic and dermopharmaceutical formulations for topical application of active ingredients to the skin.

Methods for dispersing hydrophilic active ingredients in oil are also disclosed.

### BACKGROUND OF THE INVENTION

Hydrophilic or water-soluble active ingredients are widely used in the cosmetic and pharmaceutical industry. Some ingredients are highly hydrophilic and lipophobic, meaning that they are soluble in water, but have poor solubility or no solubility in formulations with high content of oil.

However, many formulations, especially those that can be used for application of active ingredients on the skin, have a high content of oil and are highly hydrophobic. Indeed, there is a trend in some industries, like the cosmetic industry, to reduce the amount of water in formulations, and waterless products are becoming more and more common. Formulating hydrophilic active ingredients in products that are essentially hydrophobic offers an important challenge to formulators, as said products do not allow the dissolution of hydrophilic active ingredients and may compromise their stability.

Formulations using dispersions and emulsions are commonly used to solve the problem of incorporating hydrophilic active ingredients in highly hydrophobic systems.

However, such formulations often have poor stability, are chemically unstable, tend to phase separation, can have inconsistent performance, may require high amounts of emulsifiers, surfactants, or preservatives, may be opaque and not visually attractive for consumers, or do not have a good feel when applied to the skin. Different formulations comprising hydrophilic active ingredients are disclosed for instance in WO2015066877A1 and WO20221132A1.

Therefore, there exists a need to provide compositions that allow the dispersion of hydrophilic active ingredients in oil systems. There is further the need to obtain compositions that are clear or transparent and that allow active ingredients incorporated to remain stable a long time.

### SUMMARY OF THE INVENTION

The present invention solves some or all of the above-mentioned problems and provides compositions comprising hydrophilic active ingredients and can be stably formulated in oil systems. Advantageously, the compositions of the invention are clear which is more visually appealing to consumers.

In a first aspect, the invention provides a composition including:
i) from 2 to 5 wt. % of an aqueous solution comprising a hydrophilic active ingredient;
ii) from 1 to 2 wt. % of methyl glucose sesquistearate;
iii) from 10 to 12 wt % of a polyglyceryl-3 ester of a fatty acid;
iv) at least 83 wt % of an ester oil selected from the group consisting of cetyl ethylhexanoate, isopropyl isostearate and ethylhexyl palmitate; and
wherein all the weight percentages are based on the total weight of the composition; and,
wherein the sum of i), ii), iii) and iv) does not exceed 100 wt. %.

In a second aspect, the invention provides a method for dispersing a hydrophilic active ingredient in oil comprising the steps of:
(i) Dissolving a hydrophilic active ingredient in an aqueous solution; and
(ii) Mixing the aqueous solution obtained in step i) with an oil system comprising a combination of methyl glucose sesquistearate, a polyglyceryl-3 ester of a fatty acid and an ester oil selected from the group consisting of cetyl ethylhexanoate, isopropyl isostearate and ethylhexyl palmitate.

In a third aspect, the invention provides a cosmetic or dermopharmaceutical formulation which comprises the composition according to the first aspect, together with at least one cosmetically or dermopharmaceutically acceptable excipient or adjuvant.

In another aspect, the invention provides a method of cosmetic, non-therapeutic treatment and/or care of the skin, hair, nails and/or mucous membranes of a subject comprising topically administering a cosmetically effective amount of a composition according to the first aspect or a cosmetic formulation according to the third aspect.

In another aspect, the invention provides the use of a composition according to the first aspect for the cosmetic, non-therapeutic treatment and/or care of the skin, hair, nails and/or mucous membranes of a subject.

### DETAILED DESCRIPTION OF THE INVENTION

### Definitions

In the context of this invention “skin” is understood to be the layers which comprise it, from the uppermost layer or stratum corneum to the lowermost layer or hypodermis, both inclusive. These layers are composed of different types of cells such as keratinocytes, fibroblasts, melanocytes, mast cells, neurons and/or adipocytes among others. The term “skin” also comprises the scalp. The term “skin” includes the skin of mammals and includes human skin. Likewise, the terms “hair, nails and mucous membranes” include the hair, nails and mucous membranes of mammals, for example humans.

The term “treatment”, as used herein and when it is not accompanied by the qualifications “cosmetic, non-therapeutic” refers to therapeutic methods including methods directed to the administration of a compound according to the invention to alleviate or eliminate a disease or disorder, or to reduce or eliminate one or more symptoms associated with said disease or disorder. The term “treatment”, when it is not accompanied by the qualifications “cosmetic, non-therapeutic”, also covers methods of therapy directed to alleviating or eliminating physiological consequences of the disease or disorder.

When the terms “treatment” and “care” are accompanied by the qualifications “cosmetic, non-therapeutic”, it means that the treatment or care has the aim of improving or maintaining the aesthetic appearance of the skin, hair, nails and/or mucous membranes. In particular, the treatment can have the aim of improving cosmetic properties of the skin, hair, nails and/or mucous membranes such as, for example and not restricted to, the level of hydration, elasticity, firmness, shine, tone or texture, which properties affect the aesthetic appearance of the skin, hair, nails and/or mucous membranes. The term “care” in the context of this specification refers to the maintenance of properties of the skin, hair, nails and/or mucous membranes. Said properties are subject to being improved or maintained by cosmetic treatment and/or care of the skin, hair, nails and/or mucous membranes both in healthy subjects as well as in those which present diseases and/or disorders of the skin, hair, nails and/or mucous membranes.

The term “prevention”, as used in this invention, refers to the ability of a compound or a composition to prevent, delay or hinder the appearance or development of a disease or disorder, or to prevent, delay or hinder the change in a cosmetic property of the skin, mucous membranes and/or hair. The term “prevention”, as used in this invention, is interchangeable with the term “inhibition”, i.e., it refers to the ability of a compound of the invention to inhibit the appearance or development of a disease or disorder, or to inhibit the change in a cosmetic property of the skin, hair, nails and/or mucous membranes. Prevention, also relates to avoid, partially or completely, the development of a condition in subjects by taking measures to reduce the impact of the etiological causes associated with the development of such disorder. Thus, it is common to use the term prevention with the meaning of “reducing the risk of a disorder”, this is, decreasing the probability to suffering the disorder.

In the context of this invention, the term “aging” refers to the changes experienced by the skin as the result of intrinsic aging process (i.e., chronoaging) or extrinsic skin aging process induced by environmental factors (i.e., through exposure to the sun (photoaging) or to environmental agents such as tobacco smoke, extreme climatic conditions of cold or wind, chemical contaminants or pollutants). In the context of the invention, aging includes all the external visible and/or perceptible changes through touch, such as and not restricted to, the development of discontinuities on the skin such as wrinkles, fine lines, expression lines, stretch marks, furrows, irregularities or roughness, increase in the size of pores, loss of hydration, loss of elasticity, loss of firmness, loss of smoothness, loss of the capacity to recover from deformation, loss of resilience, sagging of the skin such as sagging cheeks, the appearance of bags under the eyes or the appearance of a double chin, among others, changes to the color of the skin such as marks, reddening, bags or the appearance of hyperpigmented areas such as age spots or freckles among others, anomalous differentiation, hyperkeratinization, elastosis, keratosis, hair loss, orange-peel skin, loss of collagen structure and other histological changes of the stratum corneum, of the dermis, epidermis, vascular system (for example the appearance of spider veins or telangiectasias) or of those tissues close to the skin, among others. The term “photoaging” groups together the set of processes due to the prolonged exposure of the skin to ultraviolet radiation which result in the premature aging of the skin, and it presents the same physical characteristics as aging, such as and not restricted to, flaccidity, sagging, changes to the color or irregularities in the pigmentation, abnormal and/or excessive keratinization. The sum of various environmental factors such as exposure to tobacco smoke, exposure to pollution, and climatic conditions such as cold and/or wind also contribute to the aging of the skin.

As used herein, the term “comprising”, which is inclusive or open-ended and does not exclude additional unrecited elements or method steps, is intended to encompass as alternative embodiments, the phrases “consisting essentially of” and “consisting of” where “consisting of” excludes any element or step not specified and “consisting essentially of” permits the inclusion of additional unrecited elements or steps that do not materially affect the essential or basic and novel characteristics of the composition or method under consideration.

Except in the Examples, or where otherwise explicitly indicated, all numerical quantities in this description specifying amounts of materials, reaction conditions, molecular weights, number of carbon atoms, and the like, are to be understood as modified by the word “about”. The term “about” as used herein, e.g. when referring to a measurable value (such as an amount or weight of a particular component or temperature), refers to variations of ±20%, ±10%, ±5%, ±1%, 0.5%, or, particularly, ±0.1% of the specified amount. Except where otherwise indicated, all numerical quantities in the description specifying amounts or ratios of materials are on a weight basis.

### Hydrophilic Active Ingredients

A first aspect the invention relates to a composition including:
i) from 2 to 5 wt. % of an aqueous solution including a hydrophilic active ingredient;
ii) from 1 to 2 wt. % of methyl glucose sesquistearate
iii) from 10 to 12 wt % of a polyglyceryl-3 ester of a fatty acid;
iv) at least 83 wt % of an ester oil selected from the group consisting of cetyl ethylhexanoate, isopropyl isostearate and ethylhexyl palmitate; and
wherein all the weight percentages are based on the total weight of the composition; and,
wherein the sum of i), ii), iii) and iv) does not exceed 100 wt. %. The present invention provides compositions that allow to stably formulate hydrophilic active ingredients in oil.

The compositions of the invention are suitable for topical application. Thus, in particular, the composition is a composition for topical application.

The terms “active ingredient”, “active compound” or “active substance”, as used herein, refer to a compound, which when administered in effective amounts provides a benefit to a subject, particularly a mammal, more particularly to a human subject. The active ingredient can be administered alone or in combination with other ingredients.

The term effective amount is understood to mean a non-toxic but sufficient amount of an active ingredient to provide the desired effect. For example, “cosmetically effective amount” is understood to be an amount necessary and sufficient for improving or maintaining the aesthetic appearance of the skin, hair, nails and/or mucous membranes and the term “pharmaceutically effective amount” is understood to mean an amount necessary and sufficient for preventing or treating a disease or condition. The terms “pharmaceutically effective” and “therapeutically effective” are used interchangeably herein. The effective amount of the compounds of the invention which should be administered, as well as their dosage, will depend on numerous factors, including age, state of the patient, the nature or severity of the condition, disorder or disease to be treated and/or cared for, the route, location and frequency of administration and of the particular nature of the ingredients to be used. Such an amount can be determined by the person skilled in the art by using routinary methods, for example by means of in vitro, ex vivo or in vitro trials.

The active ingredients used in compositions of this invention at cosmetically or dermopharmaceutically effective concentrations to achieve the desired effect; may be for example in amounts with respect to the total weight of the composition of: from 0.00000001% (in weight) to 20% (in weight); from 0.000001% (in weight) to 15% (in weight), from 0.00001% (in weight) to 10% (in weight); or from 0.0001% (in weight) to 5% (in weight). In particular, the active ingredient can be in an amount of at least 0.001 wt. %, more particularly from at least 0.01 wt. % based on the total weight of composition. The active ingredient may be in a concentration from 0.001 to 1 wt. %, 0.01 to 0.5 wt. %, 0.05 to 0.25 wt. % or 0.1 to 0.2 wt. % based on the total weight of composition.

Particularly, the aqueous solution comprising the hydrophilic active ingredient is in a concentration of from 2 to 3 wt. %, more particularly from 2 to 2.5 wt. % based on the total weight of the composition.

The term “hydrophilic”, as used herein, refers to the active ingredients which are soluble in distilled water (or equivalent) in at least 0.001% by weight at 25° C. at normal pressure, particularly at 0.1% by weight. Hydrophilic active ingredients are known to be lipophobic. The term “lipophobic”, as used herein, refers to the active ingredients which are not soluble in oil in at least 0.001% by weight at 25° C. at normal pressure, particularly at least by 0.1% by weight.

An advantageous feature of the present invention is that, compared to conventional water in oil emulsions, the composition is clear, or transparent. The term clear or transparent (that is clarity), according to the present invention, is intended to connote its usual dictionary definition; thus, a clear, e.g., compositions of the present invention, allow ready viewing of objects behind it. By contrast, a translucent composition allows light to pass through but causes the light to be so scattered that it will be impossible to see clearly objects behind the translucent composition. For example, optical clarity of compositions of the present invention can be measured using a turbidimeter after allowing the composition to stand for at least 24 h at room temperature, and desirably is better than 50 NTU measured at room temperature (20° C.-25° C.). Therefore, the present invention provides compositions that are more appealing for customers than conventional water-in-oil emulsions that usually have a translucent or opaque milky aspect.

The aqueous solution may contain water and small amounts of water-soluble organic solvents, particularly less than 10% by weight, or less than 5% by weight, or less than 1% by weight based on the total weight of the aqueous solution. More particularly, the aqueous solution is devoid of organic or inorganic solvents other than water, i.e. the aqueous solution only contains water as solvent, more particularly distilled water or purified water. In one embodiment, the aqueous solution consists essentially of, or even consists of, water and the hydrophilic active ingredient.

Optionally, the pH can be adjusted to fall within the range of 4.0 to 8, more particularly from 6.0 to 7.0.

Not limiting examples of hydrophilic active ingredient are humectants or substances which retain moisture, agents stimulating healing, agents stimulating re-epithelialization, agents which synthesize dermal or epidermal macromolecules, firming and/or redensifying and/or restructuring agents, cytokine growth factors, anti-glycation agents, antioxidants, free radical scavengers and/or anti-pollution agents, reactive carbonyl species scavengers, 5a-reductase-inhibiting agents, lysyl- and/or prolyl hydroxylase inhibiting agents, defensin synthesis-stimulating agents, bactericidal agents and/or bacteriostatic agents and/or antimicrobial agents and/or germicidal agents and/or fungicidal agents and/or fungistatic agents, anti-viral agents, antiparasitic agents, antihistaminic agents, desquamation agents or keratolytic agents and/or exfoliating agents, comedolytic agents, anti-psoriasis agents, anti-dandruff agents, anti-inflammatory agents and/or analgesics, anesthetic agents, anti-wrinkle and/or antiaging agents, cosmetic and/or absorbent and/or body odor masking deodorants, antiperspirant agents, agents inhibiting vascular permeability, hydrolytic epidermal enzymes, whitening or skin depigmenting agents, agents inhibiting sweat-degrading enzymes, agents capable of filtering UV rays, agents which stimulate or regulate keratinocyte differentiation, anti-itching agents, agents which stimulate or inhibit the synthesis of melanin, propigmenting agents, self-tanning agents, agents stimulating the proliferation of melanocytes, liquid propellants, vitamins and vitamin derivatives, amino acids, skin relaxant agents, agents capable of reducing or treating bags under eyes, agents for the treatment and/or care of sensitive skin, astringent agents, agents regulating sebum production, anti-stretch mark agents, lipolytic agents or agents stimulating lipolysis, venotonic agents, anti-cellulite agents, calming agents, agents to improve dermal-epidermal junction, agents inducing hair growth or hair-loss retardants, body hair growth inhibiting or retardant agents, heat shock protein synthesis stimulating agents, muscle relaxants, muscle contraction inhibitory agents, agents inhibiting the aggregation of acetylcholine receptors, anticholinergic agents, elastase inhibitory agents, matrix metalloproteinase inhibitory agents, chelating agents, mineral salts, cell extracts, emulsifying agents, agents stimulating the synthesis of lipids and components of the stratum corneum (ceramides, fatty acids, etc.), agents modulating the microbiota or microbiome of the skin, agents regulating the circadian rhythm of skin cells, skin conditioners, immunomodulators, agent improving skin luminosity or glossiness, agents for improving skin microcirculation, vascularization and/or skin oxygenation, and/or mixtures thereof.

The nature of these active ingredients can be synthetic, natural or derivatives of natural. Non-limiting examples of natural ingredients include botanical or vegetal extracts, purified ingredients obtained from botanical or vegetal extracts (i.e., phytochemical compounds), protein hydrolysates, marine extracts and ingredients obtained from biotechnological processes. In the context of this invention, biotechnological process is understood to be any process which produces the active ingredient, or part of the active ingredient, in an organism or part of it. In particular, the organism can be a microorganism. The ingredients can also be obtained from a combination of a synthetic process and a biotechnological process.

It has been surprisingly found that the combination of the components of the compositions according to the invention further improves the stability of active ingredients, such as water-soluble peptides, in oil-based formulations compared to other oil systems where the peptide is less stable and is more prone to be degraded. Therefore, of particular interest as active ingredients in the context of the invention are peptides or peptide derivatives of natural or synthetic origin.

In particular, the peptide can be a tripeptide, a tetrapeptide, a pentapeptide, a hexapeptide, a heptapeptide, an octapeptide, a nonapeptide and/or mixtures thereof.

In particular, the hydrophilic active ingredient can be one or more peptides selected from the group consisting of:

(SEQ ID. 1)
R₁-Glu-Glu-Met-Gln-Arg-Arg-R₂;
(SEQ ID. 2)
R₁-Glu-Glu-Met-Gln-Arg-Arg-Ala-Asp-R₂;
(SEQ ID. 3)
R₁-Glu-Glu-Met-Gin-Arg-Arg-Ala-R₂;
(SEQ ID. 4)
R₁-Arg-Arg-Gln-Met-Glu-Glu-R₂;
(SEQ ID. 5)
R₁-Arg-Arg-D-Gln-Met-Glu-Glu-R₂;
(SEQ ID. 6)
R₁-Arg-Arg-Gln-D-Met-Glu-Glu-R₂;
(SEQ ID. 7)
R₁-Arg-Arg-D-Gln-D-Met-Glu-Glu-R₂;
(SEQ. ID. 8)
R₁-Phe-Trp-Met-Lys-Arg-Lys-Arg-Val-Pro-R₂;
(SEQ. ID. 9)
R₁-Tyr-D-Ala-Gly-Phe-Leu-R₂;
and,
(SEQ. ID. 10)
R₁-Leu-Pro-Val-Thr-R₂;

wherein R₁ is selected from the group consisting of H and R₅—CO—, wherein R₅ is selected from the group consisting of C₁-C₁₈ alkyl, C₂-C₂₄ alkenyl, C₃-C₂₄ cycloalkyl; and R₂ is —NR₃R₄ or —OR₃ wherein R₃ and R₄ are independently selected from the group consisting of H and C₁-C₁₆ alkyl. R₁ in bound to the amino terminal end (N-terminal) of the peptide and R₂ is bound to the carboxy-terminal end (C-terminal) of the peptide.

In this description, the abbreviations used for amino acids follow the rules of IUPAC-IUB Commission of Biochemical Nomenclature specified in Eur. J. Biochem., (1984), 138, 9-37. Thus, for example, Gly represents NH₂—CH₂—COOH, Gly- represents NH₂—CH₂—CO—, -Gly represents —NH—CH₂—COOH and -Gly- represents —NH—CH₂—CO—. Therefore, the hyphen, which represents the peptide bond, eliminates the OH in the 1-carboxyl group of the amino acid (represented here in the conventional non-ionized form) when situated to the right of the symbol, and eliminates the H of the 2-amino group of the amino acid when situated to the left of the symbol; both modifications can be applied to the same symbol (see Table 1).

TABLE 1
Structures of the amino acid residues, their nomenclature in three-letter code
and nomenclature for the amino acids in one letter code

| Name | Residue | Symbol | Residue |
|---|---|---|---|
| Arginyl -Arg- R | 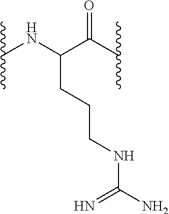 [SMILES: *NC(CCCNC(=N)N)C(*)=O] | Lysyl -Lys- K | 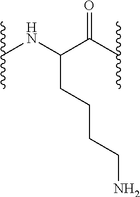 [SMILES: *NC(CCCCN)C(*)=O] |
| Glutaminyl -Gln- Q | 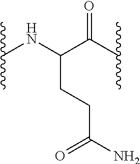 [SMILES: *NC(CCC(N)=O)C(*)=O] | Glutamyl -Glu- E | 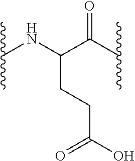 [SMILES: *NC(CCC(=O)O)C(*)=O] |
| Phenylalanyl -Phe- F | 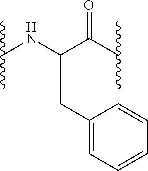 [SMILES: *NC(Cc1ccccc1)C(*)=O] | Methionyl -Met- M | 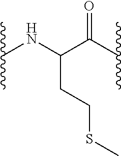 [SMILES: *NC(CCSC)C(*)=O] |
| Leucyl -Leu- L | 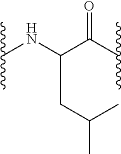 [SMILES: *NC(CC(C)C)C(*)=O] | Aspartyl -Asp- D | 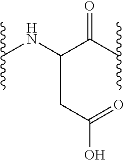 [SMILES: *NC(CC(=O)O)C(*)=O] |
| Alanyl -Ala- A | 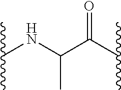 [SMILES: *NC(C)C(*)=O] | Valyl -Val- V | 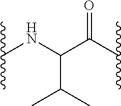 [SMILES: *NC(C(*)=O)C(C)C] |
| Tyrosyl -Tyr- Y | 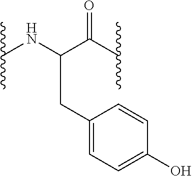 [SMILES: *NC(Cc1ccc(O)cc1)C(*)=O] | Glycyl -Gly- G | 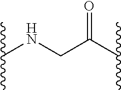 [SMILES: *NCC(*)=O] |
| Tryptophyl -Trp- W | 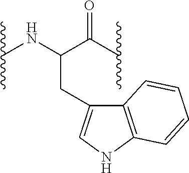 [SMILES: *NC(Cc1c[nH]c2ccccc12)C(*)=O] | Prolyl -Pro- P | 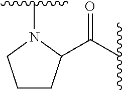 [SMILES: *C(=O)C1CCCN1*] |
| Threonyl -Thr- T | 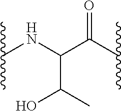 [SMILES: *NC(C(*)=O)C(C)O] | | |

As used herein, the term “alkyl” includes both saturated linear and branched alkyl groups, which may be substituted (mono- or poly-) or unsubstituted. The alkyl group is bound to the rest of the molecule by a single bond. The alkyl group has from 1 to 24, preferably from 1 to 16, more preferably from 1 to 14, even more preferably from 1 to 12, yet more preferably 1, 2, 3, 4, 5 or 6 carbon atoms. The term “alkyl” includes, for example, methyl, ethyl, isopropyl, isobutyl, tert-butyl, 2-methylbutyl, heptyl, 5-methylhexyl, 2-ethylhexyl, octyl, decyl, dodecyl, lauryl, hexadecyl, octadecyl and amyl.

As used herein, the term “alkenyl” refers to a group containing one or more double carbon-carbon bonds and which may be linear or branched and substituted (mono- or poly-) or unsubstituted. Preferably it has 1, 2 or 3 double carbon-carbon bonds. If more than one double carbon-carbon bond is present, the double bonds may be conjugated or not conjugated. Preferably the alkenyl group has from 2 to 24, preferably from 2 to 16, more preferably from 2 to 14, even more preferably from 2 to 12, yet more preferably 2, 3, 4, 5 or 6 carbon atoms. The alkenyl group is bound to the rest of the molecule by a single bond. The term “alkenyl” includes, for example, vinyl (—CH₂═CH₂), allyl (—CH₂—CH═CH₂), prenyl, oleyl, linoleyl groups and similar.

The term “cycloalkyl” refers to a saturated mono- or polycyclic alkyl group which may be substituted (mono- or poly-) or unsubstituted. The cycloalkyl group has from 3 to 24, preferably from 3 to 16, more preferably from 3 to 14, even more preferably from 3 to 12, yet even more preferably 3, 4, 5 or 6 carbon atoms. The cycloalkyl group is bound to the rest of the molecule by a single bond. Cycloalkyl groups include, for example and not restricted to, cyclopropyl, cyclobutyl, cyclopentyl, cyclohexyl, cycloheptyl, methyl cyclohexyl, dimethyl cyclohexyl, octahydroindene, decahydronaphthalene, dodecahydrophenalene and similar.

As is understood in this technical field, there may be a certain degree of substitution of the aforementioned groups. In particular, there can be substitution in any of the groups identified above where it is explicitly stated. The substituted groups (radicals) referred to above are groups (or radicals) which are substituted in one or more positions available by one or more substituents. Preferably substitution is in the 1, 2 or 3 positions, more preferably in the 1 or 2 positions, yet even more preferably in the 1 position. Suitable substituents include, for example and not restricted to: C—C₄alkyl; hydroxyl; Cr—C₄alkoxyl; amino; amino —Cr—C₄alkyl; C—C₄carbonyloxyl; CrC₄ oxycarbonyl; halogen such as fluoride, chlorine, bromine and iodine; cyano; nitro; azide; C—C₄ alkylsulfonyl; thiol; C₁-C₄ alkylthio; aryloxy such as phenoxyl; —NR_{b}(C═NR_{b})NR_{b}R_{c}; wherein R_{b} and Rₑ are independently selected from the group formed by H, C₁-C₄ alkyl, C₂-C₄alkenyl, alkynyl, C₃-C₁₀ cycloalkyl, C₆-C₁ aryl, C₇-C₁₇ aralkyl, heterocyclyl of 3-10 members or protective group of the amino group.

The amino acids which comprise the peptides can have the configuration L- or D-. In general, unless otherwise specified the amino acids are in L-configuration. For example, when the amino acid is stated to be Arg, it is understood that the amino acid is Arg or L-Arg, and when the amino acid is stated to be D-Arg the amino acid is in D-configuration.

R₁ can be selected from the group consisting of H and R₅—CO—, wherein R₅ is selected from the group consisting of C₁-C₁₈ alkyl, C₂-C₂₄ alkenyl, C₃-C₂₄ cycloalkyl or the group consisting of C₁-C₁₆ alkyl, C₂-C₁₈ alkenyl, C₃-C₇ cycloalkyl. The R₅—CO— group includes alkanoyl groups such as acetyl (CH₃—CO—, which is abbreviated herein as “Ac-”), myristoyl (CH₃-(CH₂)₁₂—CO—, which is abbreviated herein as “Myr-”) and palmitoyl (CH₃-(CH₂)₁₄—CO—, which is abbreviated herein as “Palm-”).

R₁ can be selected from the group consisting of H and acetyl, tert-butanoyl, prenyl, hexanoyl, 2-methylhexanoyl, cyclohexanecarboxyl, octanoyl, decanoyl, lauroyl, myristoyl, palmitoyl, stearoyl, oleoyl and linoleoyl.

R₁ can be selected from the group consisting of H and R₅—CO—, wherein R₅ is selected from the group consisting of C₁-C₁₆ alkyl or C₂-C₁₈alkenyl.

R₁ can be selected from the group consisting of H and R₅—CO—, wherein R₅ is C₁-C₁₅ alkyl.

R₁ can be selected from the group consisting of H, acetyl and palmitoyl.

R₂ can be —NR₃R₄ or —OR₃, wherein R₃ is H and R₄ is selected from the group formed by H and C₁-C₁₆ alkyl, including methyl, ethyl, hexyl, dodecyl and hexadecyl.

R₂ can be selected from the group consisting of —OH, —NH₂ and —NHR₄ where R₄ is C₁-C₁₆ alkyl or C₁-C₃ alkyl or C₁-C₂ alkyl.

R₂ can be —OH or —NH₂.

R₁ can be selected from the group consisting of H and R₅—CO—, wherein R₅ is selected from the group consisting of C—C₁s alkyl, C₂-C₂₄ alkenyl, C₃-C₂₄ cycloalkyl; and R₂ is —NR₃R₄ or —OR₃ wherein R₃ and R₄ are independently selected from the group consisting of H and C₁-C₁₆ alkyl. In this embodiment, R₃ can be H and R₄ can be selected from the group formed by H, C₁-C₁₆alkyl, CrC₃ alkyl and C₁-C₂ alkyl; for example, R₂ can be selected from the group consisting of —OH and —NH₂.

R₁ can be selected from the group consisting of H and acetyl, tert-butanoyl, prenyl, hexanoyl, 2-methylhexanoyl, cyclohexanecarboxyl, octanoyl, decanoyl, lauroyl, myristoyl, palmitoyl, stearoyl, oleoyl and linoleoyl; and R₂ is —NR₃R₄ or —OR₃ wherein R₃ and R₄ are independently selected from the group consisting of H and C₁-C₁₆ alkyl. In this embodiment, R₃ can be H and R₄ can selected from the group formed by H, C₁-C₁₆ alkyl, CrC₃ alkyl and C₁-C₂ alkyl; for example, R₂ can be selected from the group consisting of —OH and —NH₂.

R₁ can be selected from the group consisting of H and R₅—CO—, wherein R₅ is selected from the group consisting of C₁-C₁₆ alkyl or C₂-C₁₈alkenyl; and R₂ is —NR₃R₄ or —OR₃ wherein R₃ and R₄ are independently selected from the group consisting of H and C₁-C₁₆ alkyl. In this embodiment R₃ can be H and R₄ can be selected from the group formed by H, C₁-C₁₆ alkyl, C₁-C₃ alkyl and C₁-C₂ alkyl; for example, R₂ can be selected from the group consisting of —OH and —NH₂.

R₁ can be selected from the group consisting of H, acetyl, myristoyl or palmitoyl; and R₂ is —NR₃R₄ or —OR₃ wherein R₃ and R₄ are independently selected from the group consisting of H and C₁-C₁₆alkyl. In this embodiment R₃ can be H and R₄ can be selected from the group formed by H, C₁-C₁₆ alkyl, C₁-C₃ alkyl and C₁-C₂ alkyl; for example, R₂ can be selected from the group consisting of —OH and —NH₂.

R₁ can be selected from the group consisting of H and R₅—CO—, wherein R₅ is C₁-C₁₅ alkyl and R₂ is —NR₃R₄ or —OR₃ wherein R₃ and R₄ are independently selected from the group consisting of H and C₁-C₁₆alkyl. In this embodiment R₃ can be H and R₄ can be selected from the group formed by H, C₁-C₁₆ alkyl, Cr—C₃ alkyl and Cr—C₂ alkyl; for example, R₂ can be selected from the group consisting of —OH and —NH₂.

R₁ can be selected from the group consisting of H, acetyl and palmitoyl and R₂ is —NR₃R₄ or —OR₃ wherein R₃ and R₄ are independently selected from the group consisting of H and C₁-C₁₆ alkyl. In this embodiment R₃ can be H and R₄ can be selected from the group formed by H, C₁-C₁₆ alkyl, C₁-C₃ alkyl and Cr—C₂ alkyl; for example, R₂ can be selected from the group consisting of —OH and —NH₂.

In one embodiment, R₁ is acetyl and R₂ is NH₂.

In one embodiment, R₁ is H and R₂ is OH.

Particularly, the peptide can be selected from the group consisting of R₁-Glu-Glu-Met-Gln-Arg-Arg-R₂, R₁-Glu-Glu-Met-Gln-Arg-Arg-Ala-Asp-R₂, R₁-Arg-Arg-Gln-D-Met-Glu-Glu-R₂, R₁-Phe-Trp-Met-Lys-Arg-Lys-Arg-Val-Pro-R₂, and R₁-Tyr-D-Ala-Gly-Phe-Leu-R₂, and mixtures thereof. More particularly, the peptide can be selected from the group consisting of R₁-Glu-Glu-Met-Gln-Arg-Arg-R₂, R₁-Arg-Arg-Gln-D-Met-Glu-Glu-R₂, R₁-Tyr-D-Ala-Gly-Phe-Leu-R₂ and mixtures thereof.

The peptide can be R₁-Glu-Glu-Met-Gln-Arg-Arg-R₂. The peptide can be R₁-Arg-Arg-Gln-D-Met-Glu-Glu-R₂. The peptide can be H-Tyr-D-Ala-Gly-Phe-Leu-R₂. The peptide can be R₁-Tyr-D-Ala-Gly-Phe-Leu-R₂.

More particularly, the peptide can be one or more selected from the group consisting of:

Ac-Glu-Glu-Met-Gln-Arg-Arg-NH₂ (PEP-1);
Ac-Glu-Glu-Met-Gln-Arg-Arg-Ala-Asp-NH₂ (PEP-2);
Ac-Glu-Glu-Met-Gln-Arg-Arg-Ala-NH₂ (PEP-3);
Ac-Arg-Arg-Gin-Met-Glu-Glu-NH₂ (PEP-4);
Ac-Arg-Arg-D-Gln-Met-Glu-Glu-NH₂ (PEP-5);
Ac-Arg-Arg-Gln-D-Met-Glu-Glu-NH₂ (PEP-6);
Ac-Arg-Arg-D-Gln-D-Met-Glu-Glu-NH₂ (PEP-7);
H-Phe-Trp-Met-Lys-Arg-Lys-Arg-Val-Pro-NH₂
(PEP-8);
H-Tyr-D-Ala-Gly-Phe-Leu-OH (PEP-9);
and,
H-Leu-Pro-Val-Thr-OH (PEP-10).

Particularly, the peptide can be selected from the group consisting of PEP-1, PEP-2, PEP-6, PEP-8, PEP-9, and mixtures thereof. More particularly, the peptide can be selected from the group consisting of PEP-1, PEP-6 and PEP-9. The peptide can be PEP-1. The peptide can be PEP-6. The peptide can be PEP-9.

The cosmetically or pharmaceutically acceptable salts of the peptides in the context of the present invention are also found within the field of this invention. The term “cosmetically or pharmaceutically acceptable salt” means a salt recognized for its use in animals, for example, in mammals, and more specifically in human beings, and includes salts used to form base addition salts, either they are inorganic, for example and not restricted to, lithium, sodium, potassium, calcium, magnesium, manganese, copper, zinc or aluminum among others, or they are organic, for example and not restricted to, ethylamine, diethylamine, ethylenediamine, ethanolamine, diethanolamine, arginine, lysine, histidine or piperazine among others, or acid addition salts, either they are organic, for example and not restricted to, acetate, citrate, lactate, malonate, maleate, tartrate, fumarate, benzoate, aspartate, glutamate, succinate, oleate, trifluoroacetate, oxalate, pamoate or gluconate among others, or inorganic, for example and not restricted to, chloride, sulfate, borate or carbonate, among others. The nature of the salt is not critical, provided that it is cosmetically or pharmaceutically acceptable. The cosmetically or pharmaceutically acceptable salts of the peptides can be obtained by the conventional methods, well known in the prior art [Berge S. M. et a., “Pharmaceutical Salts”, (1977), J. Pharm. Sci., 66, 1-19].

Synthesis of the peptides and/or their cosmetically or pharmaceutically acceptable salts can be carried out according to conventional methods, known in the prior art, such as solid phase peptide synthesis methods [Stewart J. M. and Young J. D., “Solid Phase Peptide Synthesis, 2nd edition”, (1984), Pierce Chemical Company, Rockford, Illinois; Bodanzsky M. and Bodanzsky A., “The practice of Peptide Synthesis”, (1994), Springer Verlag, Berlin; Lloyd-Williams P. et al., “Chemical Approaches to the Synthesis of Peptides and Proteins”, (1997), CRC, Boca Raton, FL, USA], synthesis in solution, enzymatic synthesis [Kullmann W. “Proteases as catalysts for enzymic syntheses of opioid peptides”, (1980), J. Biol. Chem., 255(17), 8234-8238] or any combination thereof. The compounds can also be obtained by fermentation of a bacterial strain, modified or unmodified by genetic engineering with the objective of producing the desired sequences, or by controlled hydrolysis of proteins with animal or plant origins, preferably plant, which results in free peptide fragments that contain the desired sequence.

For example, a method of obtaining the peptides described in any of the embodiments above can include:
coupling of an amino acid, with the N-terminal end protected and the C-terminal end free, with an amino acid with the N-terminal end free and the C-terminal end protected or bound to a solid support;
elimination of the protective group of the N-terminal end;
repetition of the coupling sequence and elimination of the protective group of the N-terminal end until the desired peptide sequence is obtained;
elimination of the protective group of the C-terminal end or cleavage of the solid support.

Preferably, the C-terminal end is bound to a solid support and the process is carried out in solid phase and, therefore, comprises the coupling of an amino acid with the N-terminal end protected and the C-terminal end free, with an amino acid with the N-terminal end free and the C-terminal end bound to a polymeric support; elimination of the protective group of the N-terminal end; and repetition of this sequence as many times as is necessary to thus obtain the compound of desired length, finally followed by the cleavage of the synthesized compound from the original polymeric support.

The functional groups of the side chains of the amino acids are maintained conveniently protected with temporary or permanent protective groups throughout synthesis, and can be unprotected simultaneously or orthogonally to the process of cleavage of the peptide from the polymeric support.

Alternatively, solid phase synthesis can be carried out using a convergent strategy coupling a peptide with the polymeric support or with a peptide or an amino acid previously bound to the polymeric support. Convergent synthesis strategies are widely known by persons skilled in the art and are described in Lloyd-Williams P. et al., “Convergent Solid-Phase Peptide Synthesis”, (1993), Tetrahedron, 49(48), 11065-11133.

The process can comprise the additional stages of deprotection of the N-terminal and C-terminal ends and/or cleavage of the peptide from the polymeric support in an indiscriminate order, using standard procedures and conditions known in the prior art, after which the functional groups of these ends can be modified. The optional modification of the N-terminal and C-terminal ends can be carried out with the peptide of formula (1) anchored to the polymeric support or once the peptide has been separated from the polymeric support.

Optionally, R₁ can be introduced by the reaction of the N-terminal end of the compound of the invention with a R₁-X compound through a nucleophilic substitution reaction, in the presence of an adequate base and solvent, wherein the fragments that have the functional groups not involved in the N-C bond formation are suitably protected with temporary or permanent protective groups. R₁ is as defined above and X is a leaving group, for example and not restricted to, the tosyl group, the mesyl group and halogen groups among others.

Optionally and/or additionally, the R₂ radicals can be introduced by the reaction of a compound HR₂ with a complementary fragment which corresponds to the peptide of formula (1) in which R₂ is —OH in the presence of an adequate solvent and a base such as N,N-diisopropylethylamine (DIEA) or trimethylamine, or an additive such as 1-hydroxybenzotriazole (HOBt) or 1-hydroxyazabenzotriazole (HOAt), and a dehydrating agent such as a carbodiimide, a uronium salt, a phosphonium salt or amidinium salt, among others, or by prior formation of an acyl halide with, for example, thionyl chloride, and thereby obtaining a peptide according to the invention of formula (I), wherein the fragments that have the functional groups not involved in the N-C bond formation are suitably protected with temporary or permanent protective groups. Alternatively other R₂ radicals may be introduced by simultaneous incorporation to the peptide cleavage process from the polymeric carrier. R₂ is —OR₃, —NR₃R₄ or —SR₃, where R₃ and R₄ are as defined above.

A person skilled in the art would easily understand that the deprotection/cleavage steps of the C-terminal and N-terminal ends and their subsequent derivatization can be performed in a different order, according to the processes known in the prior art.

The term “protective group” relates to a group which blocks an organic functional group and which can be removed in controlled conditions. The protective groups, their relative reactivities and the conditions in which they remain inert are known to the person skilled in the art.

Examples of representative protective groups for the amino group are amides, such as amide acetate, amide benzoate, amide pivalate; carbamates such as benzyloxycarbonyl (Cbz or Z), 2-chlorobenzyl (CIZ), para-nitrobenzyloxycarbonyl (pNZ), tert-butyloxycarbonyl (Boc), 2,2,2-trichloroethyloxycarbonyl (Troc), 2-(trimethylsilyl)ethyloxycarbonyl (Teoc), 9-fluorenylmethyloxycarbonyl (Fmoc) or allyloxycarbonyl (Alloc), trityl (Trt), methoxytrityl (Mtt), 2,4-dinitrophenyl (Dnp), N-[1-(4,4-dimethyl-2,6-dioxocyclohex-1-ylidene)ethyl (Dde), 1-(4,4-dimethyl-2,6-dioxo-cyclohexylidene)-3-methylbutyl (ivDde), 1-(1-adamantyl)-1-methylethoxycarbonyl (Adpoc), among others, preferably Boc or Fmoc.

Examples of representative protective groups for the carboxyl group are esters, such as the tert-butyl ester (tBu), allyl ester (All), triphenylmethyl ester (Trt tester), cyclohexyl ester (cHx), benzyl ester (Bzl), ortho-nitrobenzyl ester, para-nitrobenzyl ester, para-methoxybenzyl ester, trimethylsilylethyl ester, 2-phenylisopropyl ester, fluorenylmethyl ester (Fm), 4-(N-[1-(4,4-dimethyl-2,6-dioxocyclohexylidene)-3-methylbutyl]amino) benzyl ester (Dmab), among others; preferred protective groups of the invention are the All, tBu, cHex, Bzl and Trt esters.

The side chains of trifunctional amino acids can be protected during the synthetic process with temporary or permanent protective groups orthogonal to the protective groups of the N-terminal and C-terminal ends.

The hydroxyl group of the tyrosine side chain can be protected with the 2-bromobenzyloxycarbonyl group (2-BrZ), tBu, All, Bzl or 2,6-dichlorobenzyl (2,6-diClZ) among others. In a preferred embodiment, the protective group strategy used is the strategy wherein the amino groups are protected by Boc, the carboxyl groups are protected by Bzl, cHx or All esters and the tyrosine side chain is protected with 2-BrZ or Bzl. In another preferred embodiment, the protective group strategy used is the strategy wherein the amino groups are protected by Fmoc, the carboxyl groups are protected by tBu, All or Trt esters, the tyrosine side chain is protected by tBu.

The amino group of the tryptophan side chain can be protected, for example, by the formyl group (For) or Boc. In one embodiment, when the amino group is protected by Fmoc, and the tryptophan side chain can be: unprotected, i.e., the amino acid is incorporated as Fmoc-Trp-OH; protected by Boc, i.e. the amino acid is incorporated as Fmoc-Trp(Boc)-OH; or protected by For, i.e., the amino acid is incorporated as Fmoc-Trp(For)-OH. In one embodiment, the amino group is protected by Boc, and the tryptophan side chain can be protected by For, i.e., the amino acid is incorporated as Boc-Trp(For)-OH.

Examples of these and other protective groups, their introduction and removal, can be found in the literature [Atherton B. and Sheppard R. C., “Solid Phase Peptide Synthesis: A practical approach”, (1989), IRL Oxford University Press]. The term “protective groups” also includes the polymeric supports used in solid phase synthesis.

When synthesis takes place totally or partially in solid phase, the possible solid supports used in the process of the invention involve polystyrene support, polyethylene glycol grafted to polystyrene and similar, for example and not restricted to, p-methylbenzhydrylamine resins (MBHA) [Matsueda G. R. et al., “A p-methylbenzhydrylamine resin for improved solid-phase synthesis of peptide amides”, (1981), Peptides, 2, 45-50], 2-chlorotrityl resins [Barlos K. et al., “Darstellung geschützter Peptid-Fragmente unter Einsatz substituierter Triphenylmethyl-Harze”, (1989), Tetrahedron Lett., 30, 3943-3946; Barlos K. et al., “Veresterung von partiell geschutzten Peptid-Fragmenten mit Harzen. Einsatz von 2-Chlorotritylchlorid zur Synthese von Leu1-Gastrin I”, (1989), Tetrahedron Lett., 30, 3947-3951], TentaGel® resins (Rapp Polymere GmbH), ChemMatrix™ resins (Matrix Innovation, Inc) and similar, which may or may not include a labile linker, such as 5-(4-aminomethyl-3,5-dimethoxyphenoxy) valeric acid (PAL) [Albericio, F. et al., “Preparation and application of the 5-(4-(9-fluorenylmethyloxycarbonyl) aminomethyl-3,5-dimethoxy-phenoxy)valeric acid (PAL) handle for the solid-phase synthesis of C-terminal peptide amides under mild conditions”, (1990), J. Org. Chem., 55, 3730-3743], 2-[4-aminomethyl-(2,4-dimethoxyphenyl)]phenoxyacetic acid (AM) [Rink H., “Solid-phase synthesis of protected peptide fragments using a trialkoxy-diphenyl-methylester resin”, (1987), Tetrahedron Lett., 28, 3787-3790], [Wang S. S., “p-Alkoxybenzyl Alcohol Resin and p-Alkoxybenzyloxycarbonylhydrazide Resin for Solid Phase Synthesis of Protected Peptide Fragments”, (1973), J. Am. Chem. Soc., 95, 1328-1333] and similar, which enable simultaneous deprotection and cleavage of the compound from the polymeric support.

### Methyl Glucose Sesquistearate

The invention provides compositions comprising at least a methyl glucose sesquistearate. Methyl glucose sesquistearate is a mixture of mono- and diesters of a methyl glucoside and stearic acid.

A non-limiting example of methyl glucose sesquistearate useful in the context of this invention includes Glucate™ SS emulsifier from Lubrizol Advanced Materials, Inc.

The methyl glucose sesquistearate can be in a concentration from 1 to 2 wt. % based on the total weight of the composition, particularly from 1 to 1.5 wt. %. Particularly, can be in a concentration of 1, 1.1, 1.2, 1.4 or 1.5 wt. % based on the total weight of the composition.

### Polyglyceryl-3 Ester of Fatty Acid

The invention provides compositions comprising at least an ester of fatty acids. In particular, the compositions of the invention comprise at least one polyglyceryl-3 ester of fatty acid. These can be described by the following formula:

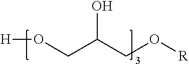

in which R stands for the acyl radical of a fatty acid.

Polyglyceryl-3 esters of fatty acids particularly useful for the context of this invention are polyglyceryl-3 caprylate, polyglyceryl-3 caprate, polyglyceryl-3 laurate, polyglyceryl-3 myristate, polyglyceryl-3 palmitate, polyglyceryl-3 stearate, polyglyceryl-3 arachinate, polyglyceryl-3 behenate, polyglyceryl-3 palmitoleate, polyglyceryl-3 oleate, polyglyceryl-3 linoleate. In particular, the polyglyceryl-3 ester of fatty acid is selected from the group consisting of polyglyceryl-3 caprylate, polyglyceryl-3 caprate, polyglyceryl-3 laurate, polyglyceryl-3 myristate and polyglyceryl-3 palmitate. More particularly, the polyglycerol-3 ester of fatty acid can be polyglyceryl-3 laurate, also referred as triglycerol laurate. A non-limiting example of triglycerol laurate useful of the context of this invention is Hydramol™ TGL ester from Lubrizol Advanced Materials, Inc.

The polyglycerol-3 ester of fatty acid can be in a concentration from 10 to 12 wt. % based on the total weight of the composition. In particular, the concentration can be 10, 11 or 12 wt. % based on the total weight of the composition.

### Ester Oil

The compositions of the invention further comprise an ester oil. Particularly, the ester oil is selected from the group consisting of cetyl ethylhexanoate, isopropyl isostearate, ethylhexyl palmitate and mixtures thereof.

Examples of these ingredients that may be mentioned are Schercemol™ CO ester from Lubrizol Advanced Materials, Inc. (cetyl ethylhexanoate), Schercemol™ 318 ester (isopropyl isostearate) from Lubrizol Advanced Materials, Inc., and DUB™ PO (ethylhexyl palmitate) from Stéarinerie Dubois.

Advantageously, cetyl ethylhexanoate has been found to allow obtaining compositions completely free of precipitate, which is more visually appealing for the consumer. Thus, particularly, the ester oil can be cetyl ethylhexanoate.

The ester oil can be in a concentration from at least 83 wt. % based on the total weight of the composition. The ester oil can be in a concentration from 85 to 87 wt. %, or from 86 to 87 wt. % based on the total weight of the composition.

In particular, the invention provides compositions comprising from 2 to 3 wt. % of an aqueous solution comprising a hydrophilic active ingredient, particularly a peptide, from 1 to 2 wt. % of methyl glucose sesquistearate, from 10 to 12 wt. % of a polyglyceryl-3 ester of a fatty acid and from 85 to 87% by weight of an ester oil selected from the group consisting of cetyl ethylhexanoate, isopropyl isostearate and ethylhexyl palmitate; wherein all the weight percentages are based on the total weight of the composition; and, wherein the sum of i), ii), iii) and iv) does not exceed 100 wt. %.

In particular, the invention provides compositions comprising from 2 to 3 wt. % of an aqueous solution comprising a hydrophilic active ingredient, particularly a peptide, from 1 to 2 wt. % of methyl glucose sesquistearate, from 10 to 12 wt. % of triglycerol laurate and from 85 to 87% by weight of an ester oil selected from the group consisting of cetyl ethylhexanoate, isopropyl isostearate and ethylhexyl palmitate; wherein all the weight percentages are based on the total weight of the composition; and, wherein the sum of i), ii), iii) and iv) does not exceed 100 wt. %.

In particular, the invention provides compositions comprising from 2 to 3 wt. % of an aqueous solution comprising a hydrophilic active ingredient, particularly a peptide, from 1 to 2 wt. % of methyl glucose sesquistearate, from 10 to 12 wt. % of a polyglyceryl-3 ester of a fatty acid and from 85 to 87% by weight of cetyl ethylhexanoate; wherein all the weight percentages are based on the total weight of the composition; and, wherein the sum of i), ii), iii) and iv) does not exceed 100 wt. %.

In particular, the invention provides compositions comprising from 2 to 3 wt. % of an aqueous solution comprising a hydrophilic active ingredient, particularly a peptide, from 1 to 2 wt. % of methyl glucose sesquistearate, from 10 to 12 wt. % of triglycerol laurate and from 85 to 87% by weight of cetyl ethylhexanoate; wherein all the weight percentages are based on the total weight of the composition; and, wherein the sum of i), ii), iii) and iv) does not exceed 100 wt. %.

### Cosmetic and Dermopharmaceutical Formulations

The compositions of the invention can be administered for their application by any means that causes contact between the compounds and the site of action in a subject's body, preferably that of a mammal, preferably a human, and in the form of a cosmetic or dermopharmaceutical formulation or product which contains them, together with cosmetically or dermopharmaceutically acceptable excipients or adjuvants.

The terms “cosmetically acceptable excipient” and “dermopharmaceutically acceptable excipient” are well known by the person skilled in the art and includes compounds, materials, compositions, carriers, vehicles and/or dosage forms which, when mixed the compositions, improve the application on the skin without excessive toxicity, irritation, allergic response, or other problem or complication, commensurate with a reasonable benefit/risk ratio. Suitable excipients or adjuvants can be found in standard cosmetic or pharmaceutical texts known to persons skilled in the art such as for example Harry's Cosmeticology”, Seventh edition, (1982), Wilkinson J. B., Moore R. J., ed. Longman House, Essex, GB or “Handbook of Pharmaceutical Excipients” Raymond C Rowe, Paul J Sheskey and Sian C Owen, Royal Pharmaceutical Society of Great Britain London, UK.

The composition according to the first aspect of the invention can be included in a cosmetic or dermopharmaceutical formulation in an amount from 0.001 to 10 wt. %, particularly from 0.01 to 5 wt. %, more particularly from 0.1 to 2 wt. %, based on the total weight of the cosmetic or dermopharmaceutical formulation.

The cosmetic or dermopharmaceutical formulations for topical or transdermal application can be produced in any solid, liquid or semisolid formulation, such as and not restricted to, creams, multiple emulsions such as and not restricted to, oil and/or silicone in water emulsions, water-in-oil and/or silicone emulsions, water/oil/water or water/silicone/water type emulsions and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, sticks, pencils and sprays or aerosols (sprays), including leave-on and rinse-off formulations.

Particularly, the invention provides for a cosmetic or dermopharmaceutical formulation in the form of cream, multiple emulsion, foundation, solution, anhydrous formulation, dispersion, oil, milk, balsam, lotion, gel, cream gel, liniment, conditioner, serum, stick, ointment, mousse, pomade, powder, bar, pencil, spray, aerosol, or mask.

The cosmetic or dermopharmaceutical formulation can also be incorporated into fabrics, non-woven fabrics and medical or cosmetic devices which are in direct contact with the skin. Examples of fabrics, non-woven fabrics, garments, medical or cosmetic devices and means for immobilizing the compounds to them, among which are the delivery systems and/or the sustained release systems described above, can be found in literature and are known in the prior art [Schaab C. K. (1986) HAPPI May 1986; Nelson G., “Application of microencapsulation in textiles”, (2002), Int. J. Pharm., 242(1-2), 55-62; “Biofunctional Textiles and the Skin” (2006) Curr. Probl. Dermatol. v.33, Hipler U. C. and Elsner P., eds. S. Karger A G, Basel, Switzerland; Malcolm R. K. et al., “Controlled release of a model antibacterial drug from a novel self-lubricating silicone biomaterial”, (2004), J. Cont. Release, 97(2), 313-320]. The preferred fabrics, non-woven fabrics, garments and medical devices are bandages, gauzes, t-shirts, socks, tights, underwear, girdles, gloves, diapers, sanitary napkins, dressings, bedspreads, wipes, adhesive patches, non-adhesive patches, occlusive patches, microelectric patches and/or face masks.

The cosmetic or dermopharmaceutical formulations can include saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C₁₀ to C₂₂, long chain fatty amines from about C₁₀ to C₂₂, fatty alcohols, ethoxylated fatty alcohols and di-tail phospholipids.

Representative saccharides include nonionic or cationic saccharides such as agarose, amylopectins, amyloses, arabinans, arabinogalactans, arabinoxylans, carrageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitins, chitins, chitosan, chitosan pyrrolidone carboxylate, chitosan glycolate chitosan lactate, cocodimonium hydroxypropyl oxyethyl cellulose, colominic acid (poly-N acetyl-neuraminic acid), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomannans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragacanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, starch hydroxypropyltrimonium chloride, hydroxypropyl starch phosphate, steardimonium hydroxyethyl cellulose, raffinose, rhamsan, tapioca starch, welan, levan, scleroglucan, sodium alginate, stachyose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994) which is incorporated in its entirety by reference. Complex carbohydrates can be found for example in Kirk-Othmer Encyclopedia of Chemical Technology, Fourth Edition, Vol. 4, John Wiley and Sons, NY pp. 930-948, 1995. More particularly, the formulations may comprise microcrystalline cellulose. One suitable microcrystalline cellulose is Vivapur® CS 9 GM from JRS—J. Rettenmaier & S5hne GmbH & Co. KG.

The cosmetic or dermopharmaceutical formulations of this invention may include surface-active agents. Surface active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents. Anionic surface-active agents useful herein include those disclosed in U.S. Pat. No. 5,573,709, incorporated herein by reference.

The anionic, cationic, nonionic, amphoteric or zwitterionic surface-active agents used in the cosmetic or dermopharmaceutical composition of this invention are typically used in an amount from about 0.1 to 50 percent by weight, preferably from about 0.5 to about 40 percent by weight, more preferably from about 1 to about 20 percent by weight.

The cosmetic or dermopharmaceutical formulations of this invention may also include fatty alcohols (typically monohydric alcohols), ethoxylated fatty alcohols, and di-tail phospholipids, which can be used to stabilize emulsion or dispersion forms of the cosmetically acceptable compositions. They also provide a cosmetically or pharmaceutically acceptable viscosity. Examples include stearyl alcohol, cetyl alcohol, cetostearyl alcohol, myristyl alcohol, behenyl alcohol, arachidic alcohol, isostearyl alcohol, and isocetyl alcohol.

The cosmetically or dermopharmaceutical formulations of this invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant may be present in the formulation at concentrations of from about 0.5 to about 40 percent by weight, particularly from about 0.5 to about 20 percent by weight and more particularly from about 0.5 to about 12 percent by weight.

The cosmetic or dermopharmaceutical formulations of this invention may include petrolatum or mineral oil components, which when selected will generally be USP or NF grade. Petrolatum can be partially replaced with mixtures of hydrocarbon materials, which can be formulated to resemble petrolatum in appearance and consistency. For example, mixtures of petrolatum or mineral oil with different waxes and the like may be combined. Particular waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, Vaseline® solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Corning®, Midland, Mich., USA. Additional alkyl and phenyl silicones may be employed to enhance moisturizing properties. Resins such as dimethicone (and) Trimethylsiloxysilicate, known as Dow Corning® 593 or Cyclomethicone (and) Trimethylsiloxysilicate, known as Dow Corning® 749 fluid, may be utilized to enhance film formation of skin care products. When used, the petrolatum, wax or hydrocarbon or oil component is included in the formulations at a concentration of about 0.1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetic and dermopharmaceutical formulations of this invention may include fatty ester emollients, which are listed in the International Cosmetic Ingredient Dictionary, Eighth Edition, 2000, p. 1768 to 1773.

Specific examples of suitable fatty esters for use in the formulations of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C12 to C16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, caprylic/capric triglycerides, and isostearyl isostearate. When used the fatty ester emollient is preferably included in the formulations of this invention at a concentration of about 0.1 to about 99 weight percent, more preferably about 1 to about 40 weight percent.

The cosmetic or dermopharmaceutical formulations of this invention may also include silicone compounds. Preferably, the viscosity of the silicone component at a temperature of 25° C. is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane endblocked with trimethyl units, is one preferred example. Dimethicone having a viscosity between 50 and 1,000 cps is particularly preferred. When used, the silicone oils are preferably included in the formulations of this invention at a concentration of 0.1 to 15 weight percent, more preferably 0.5 to 5 weight percent.

The cosmetic or dermopharmaceutical formulations of this invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane, available under the tradename Dow Corning® 200 fluid having a viscosity of about 1 centistoke. When used, the silicone oils are preferably included in the formulations of this invention at a concentration of 0.1 to 30 weight percent, more preferably 1 to 20 weight percent.

The cosmetic and dermopharmaceutical formulations of this invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., USA under the tradenames Dow Corning® 244 fluid, Dow Corning® 245 fluid, Dow Corning® 246, Dow Corning® 344 fluid and Dow Corning® 345 fluid, and Silicone SF-1173 and Silicone SF-1202 from General Electric, Waterford, N.Y., USA. When used, the silicone oils are preferably included in the formulations of this invention at a concentration of 0.1 to 30 weight percent, more preferably 1 to 20 weight percent. Silicone surfactants or emulsifiers with polyoxyethylene or polyoxypropylene side chains may also be used in the formulations of the current invention. Preferred examples include dimethicone copolyols, Dow Corning® 3225C and 5225C Formulation Aids, available from Dow Corning, Midland, Mich., USA and Silicone SF-1528™, available from General Electric, Waterford, N.Y., USA. The side chains may also include alkyl groups such as lauryl or cetyl. Preferred are lauryl methicone copolyol, known as Dow Corning® 5200 Formulation Aid, and cetyl dimethicone copolyol, known as Abil EM-90™, available from Goldschmidt Chemical Corporation, Hopewell, Va. Also preferred is lauryl dimethicone, known as Belsil LDM 3107 VP™, available from Wacker-Chemie, Munchen, GER. When used, the silicone surfactants are preferably included in the formulations of this invention at a concentration of 0.1 to 15 weight percent, more preferably 1 to 5 weight percent. Amine functional silicones and emulsions may be utilized in the present invention. Preferred examples include Dow Corning® 8220, Dow Corning® 939, Dow Corning® 949, Dow Corning® 2-8194, all available from Dow Corning, Midland, Mich., USA. Also preferred is Silicone SM253™ available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetic or dermopharmaceutical formulations may include volatile hydrocarbon oils. The volatile hydrocarbon comprises about C6 to C22 atoms. A preferred volatile hydrocarbon is an aliphatic hydrocarbon having a chain length of about C6 to C16 carbon atoms. An example of such compound includes isohexadecane, under the tradename Permethyl 101A™, available from Presperse, South Plainfield, N.J., USA. Another example of a preferred volatile hydrocarbon is a C12 to C14 isoparaffin, under the tradename Isopar M™, available from Exxon, Baytown, Tex., USA. In one embodiment the hydrocarbon is a C9-12 alkane. One example is SilSense™ Bio 5 emollient from Lubrizol Advanced Materials, Inc. When used, the volatile hydrocarbons are preferably included in the formulations of this invention at a concentration of 0.1 to 99 weight percent, more preferably 1 to 40 weight percent.

The cosmetic or dermopharmaceutical formulations may include cationic and ampholytic conditioning polymers. Examples of such polymers include, but are not limited to, those listed by the International Cosmetic Ingredient Dictionary published by the Cosmetic, Toiletry, and Fragrance Association (CTFA), 1101 17th Street, N. W., Suite 300, Washington, D.C. 20036. General examples include quaternary derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquatemium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, Polyquaternium-55 and mixtures thereof. When used, the conditioning polymers are preferably included in the cosmetically acceptable composition of this invention at a concentration of from 0.1 to 10 weight percent, preferably from 0.2 to 6 weight percent and most preferably from 0.2 to 5 weight percent.

The cosmetic or dermopharmaceutical formulations of this invention may include one or more rheological modifiers. The rheological modifiers which can be used in this invention include, but are not limited to high molecular weight crosslinked homopolymers of acrylic acid, and Acrylates/C10-30 Alkyl Acrylate Crosspolymer, such as the Carbopol® and Pemulen® series, both available from Lubrizol Advanced Materials, Inc.; anionic acrylate polymers such as Salcare® AST and cationic acrylate polymers such as Salcare® SC96, available from Ciba Specialties, High Point, N.C., USA; acrylamidopropyltrimonium chloride/acrylamide; hydroxyethyl methacrylate polymers, Steareth-10 Allyl Ether/Acrylate Copolymer; Acrylates/Beheneth-25 Methacrylate Copolymer, known as Aculyn® 28, available from Rohm and Haas/International Specialties, Wayne, N.J., USA; glyceryl polymethacrylate, Acrylates/Steareth-20 Methacrylate Copolymer; bentonite; gums such as alginates, carrageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof. When used, the rheology modifiers are preferably included in formulations of this invention at a concentration of from 0.01 to 12 weight percent, preferably from 0.05 to 10 weight percent and most preferably from 0.1 to 6 weight percent.

The cosmetic or dermopharmaceutical formulations of this invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The antioxidants will be present at from 0.01 to 5 weight percent, preferably 0.1 to 3 weight percent and most preferably from 0.2 to 2 weight percent of the formulation. The cosmetic or dermopharmaceutical formulations of this invention may include one or more preservatives. Examples of preservatives include, but are not limited to, 1,2-dibromo-2,4-dicyano butane (Methyldibromo Glutaronitrile, known as MERGUARD®, Ondeo Nalco Company, Naperville, Ill., USA), benzyl alcohol, imidazolidinyl urea, 1,3-bis(hydroxymethyl)-5,5-dimethyl-2, 3-imidazolidinedione (e.g., DMDM Hydantoin, known as GLYDANT®, Lonza, Fairlawn, N.J., USA.), methylchloroisothiazolinone and methylisothiazolinone (e.g., Kathon®, Rohm & Haas Co., Philadelphia, Pa., USA), methyl paraben, propyl paraben, phenoxyethanol, and sodium benzoate, and mixtures thereof.

The cosmetic or dermopharmaceutical formulations of this invention may include any other ingredient normally used in cosmetics. Examples of such ingredients include, but are not limited to, buffering agents, fragrance ingredients, chelating agents, pigments, color additives or dyestuffs which can serve to color the composition itself or keratin, sequestering agents, softeners, foam synergistic agents, foam stabilizers, sun filters, sunscreens, peptizing agents electrolytes such as aluminum chlorohydrate or alkali metal salts, or salts with organic acids.

The cosmetic or dermopharmaceutical formulations of this invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerine, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture. These solvents can be present in proportions of up to as much as 70 percent by weight, for example from 0.1 to 70 percent by weight, relative to the weight of the total composition.

The cosmetic or dermopharmaceutical formulations of the invention may include agents which increase the percutaneous absorption of active ingredients, for example and not restricted to, dimethylsulfoxide, dimethylacetamide, dimethylformamide, surfactants, azone (1-dodecylazacycloheptane-2-one), alcohol, urea, ethoxydiglycol, acetone, propylene glycol or polyethylene glycol, among others. Furthermore, the cosmetic or dermopharmaceutical formulations of this invention can be applied to local areas to be treated by means of iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof, to achieve a greater penetration of active ingredients. The application area will be determined by the nature of the condition, disorder and/or disease to be treated and/or cared for.

The cosmetic or dermopharmaceutical formulations of the invention may include additional active ingredients which can be found in solution or dispersion. Non-limiting examples include amino acids, peptides, proteins, hydrolyzed proteins, enzymes, hormones, vitamins, mineral salts, sugars, nucleotides, nucleic acids, molecules and extracts of biological and biotechnological origin, vegetable extracts, cell extracts, essential oils, molecules of synthetic or natural origin, isoflavones, polyphenols, retinol, hyaluronic acid and/or mixtures thereof. In particular, the cosmetic or dermopharmaceutical formulation may contain dimethylmethoxy chromanol, e.g., Lipochroman™ molecule from Lipotec S. A.

Particularly, the cosmetic or dermopharmaceutical formulations of the invention may comprise, in addition of the compositions of the invention, a C9-12 alkane and/or a coco-caprylate/caprate.

Particularly, the cosmetic or dermopharmaceutical formulations of the invention may comprise, in addition of the compositions of the invention, a C9-12 alkane, a coco-caprylate/caprate, AlgaPur™ High Stability High Oleic (HSHO) Algae Oil, a jojoba seed oil, tocopheryl acetyl acetate, methyl glucose dioleate and/or mixtures thereof. Optionally, the formulation may comprise fragrances and/or antioxidants. More particularly, the formulation comprises a C9-12 alkane, a coco-caprylate/caprate, AlgaPur™ High Stability High Oleic (HSHO) Algae Oil, a jojoba seed oil, tocopheryl acetyl acetate and methyl glucose dioleate.

Particularly, the cosmetic or dermopharmaceutical formulations of the invention may comprise, in addition to the compositions of the invention, a C9-12 alkane, coco-caprylate/caprate, dimethicone/vinyl dimethicone crosspolymer, and/or mixtures thereof. More particularly, the formulation may further include fragrances and/or preservatives.

Particularly, the cosmetic or dermopharmaceutical formulations of the invention may comprise, in addition of the compositions of the invention, a C9-12 alkane, coco-caprylate/caprate, silica dimethyl silylate, dimethicone/vinyl dimethicone crosspolymer, polyglyceryl-3 polyricinoleate, methyl glucose dioleate, polyhydroxystearic acid, neopentyl glycol diethylhexanoate, isododecane, disteardimonium hectorite, propylene carbonate, titanium dioxide, iron oxides, propanediol, glyceryl caprylate, magnesium sulfate, and sodium benzoate. More particularly, the formulation may further include fragrances and/or preservatives.

Particularly, the cosmetic or dermopharmaceutical formulations of the invention may comprise, in addition of the compositions of the invention, caprylic/capric triglyceride, neopentyl glycol diethylhexanoate, isostearyl isostearate and/or mixtures thereof. More particularly the formulations may comprise, caprylic/capric triglyceride, polyurethane-79, neopentyl glycol diethylhexanoate, isostearyl isostearate, silica, wax, and glyceryl caprylate. More particularly the wax is carnauba wax, candelilla wax, bran wax and/or mixtures thereof.

Particularly, the cosmetic or dermopharmaceutical formulations of the invention may comprise, in addition to the compositions of the invention, isododecane, isostearyl isostearate, microcrystalline cellulose, at least one C9-C12 alkane and/or mixtures thereof.

### Methods

In one aspect the invention provides a method for dispersing a hydrophilic active ingredient in oil comprising the steps of:
(i) Dissolving a hydrophilic active ingredient in an aqueous solution; and
(ii) Mixing the aqueous solution obtained in step i) with an oil system comprising a combination of methyl glucose sesquistearate, a polyglyceryl-3 ester of a fatty acid and an ester oil selected from the group consisting of cetyl ethylhexanoate, isopropyl isostearate and ethyl disteardimonium hectorite, ethyl palmitate.

In particular, the hydrophilic active ingredient can be dissolved in the aqueous solution in a concentration of from 0.1 to 100 mg/mL, or from 1 to 10 mg/mL, or from 2.5 to 7.5 mg/ml.

In particular, the aqueous solution consists essentially of, or even consists of, water. More particularly, the water is distilled or purified water.

In particular, the oil system (ii) is heated at 40-70° C., particularly, 50-65° C., before mixing with the aqueous solution comprising the hydrophilic active ingredient of i).

In particular, the aqueous solution comprising the hydrophilic active ingredient of i) is slowly added on top of the oil system (ii) with continuous mechanic stirring and the resultant mixture is mixed with continuous mechanic stirring until the temperature cool down to 20-25° C.

The polyglyceryl-3 ester can be particularly triglycerol laurate.

The ester oil can be particularly cetyl ethylhexanoate.

Thus, in one embodiment the invention provides a method for dispersing a hydrophilic active ingredient in oil comprising the steps of (i) Dissolving a hydrophilic active ingredient in an aqueous solution; and (ii) Mixing the aqueous solution obtained in step i) with an oil phase comprising a combination of methyl glucose sesquistearate, a triglycerol laurate and cetyl ethylhexanoate. More particularly, the oil phase is heated at 40-70° C. before mixing, preferably at 50-65° C.

### Applications

The compositions described herein are useful to deliver hydrophilic active ingredients for the treatment of the skin, hair, nails and/or mucous membranes. The compositions are particularly useful for the delivery of hydrophilic cosmetic active ingredients.

Thus, in one aspect the invention provides for a method of cosmetic, non-therapeutic treatment and/or care of the skin, hair, nails and/or mucous membranes of a subject comprising topically administering a cosmetically effective amount of a composition or cosmetic formulation according to the previous aspects.

Non-limiting examples of cosmetic, non-therapeutic treatment and/or care in the context of this invention include the treatment and/or prevention of the aging of the skin, the treatment and/or prevention of skin wrinkles; maintaining and improving skin firmness; the stimulation of collagen synthesis and/or prevention of collagen loss; the treatment and/or prevention of a sagging appearance of the skin; the reduction and/or prevention of facial asymmetry; the increase of the volume of adipose tissue; and/or the prevention and/or alleviation of the effects of adipose tissue loss; treatment and/or prevention of photoaging of the skin; promoting the self-renewal properties of the skin; modulation of skin microbiota; prevention and/or treatment of dandruff; maintenance and/or improvement of skin hydration and/or moisturization; maintenance and/or improvement of skin microcirculation; maintenance and/or improvement of skin lightening and/or brightening; whitening the skin and/or reduction of black spots; treatment and/or prevention of skin fatigue; maintenance and/or improvement of skin barrier function; maintenance and/or improvement of skin tone; and/or energizing the skin.

In particular, the method of cosmetic, non-therapeutic treatment and/or care can be the treatment and/or prevention of skin aging; and/or the reduction and/or prevention of skin wrinkles.

The invention also provides the use of a composition according to the first aspect for the cosmetic, non-therapeutic treatment and/or care of the skin, hair, nails and/or mucous membranes of a subject. Particularly, the use is for the treatment and/or prevention of skin aging; and/or the reduction and/or prevention of skin wrinkles.

### EXAMPLES

The invention is illustrated by the following non-limiting examples. These examples are included solely for illustrative purposes and should not be construed as limitations on the invention claimed herein.

### Examples 1 to 5

Different compositions containing PEP-1 were prepared comprising the ingredients set out in Table 2.

The procedure consisted of mixing two phases: water phase (Phase A) and oil phase (Phase B). Phase A is a solution to the peptide in water. Phase B is the dispersion of Glucate™ DO emulsifier from Lubrizol Advanced Materials, Inc. and Algapur™ High Stability High Oleic (HSHO) Algae Oil from Lubrizol Advanced Materials, Inc. Phase A is added into phase B slowly with mixing.

The stability of the formulations was determined by visual inspection and by quantification of the amount of active ingredient by high performance liquid chromatography (HPLC).

Visual inspection: The results are classified as OK or KO according to the physical appearance. OK represents a transparent solution at t=0 h with small particles at the bottom after 24h. KO represents solutions completely separated into phases, turbid or with an unacceptable precipitate at the bottom.

Stability: The concentration of the peptide was determined by HPLC after storing the compositions for 1 week at room temperature. Compositions were 10 times diluted in 1 g of dimethyl sulfoxide (DMSO). Twenty-five microliters of the solution are then injected in a Kromasil™ 100 Å, C-8, 5 μm., 250×4.6 mm column. The solvents used are: Solvent A, H₂O/TFA and Solvent B, acetonitrile/TFA.

The separation gradient was:

| Time (min) | [Solvent B] (%) |
|---|---|
| 0 | 5 |
| 30 | 35 |
| 32 | 100 |
| 42 | 100 |

Concentration of peptide was determined by correlating the sum of the areas of the peaks corresponding to non-oxidized and oxidized peptide with that of a standard of the peptide.

The recovery of peptide was calculated as:

$$Recovery ( \% ) = \frac{Peptide concentration of peptide as determined by HPLC ( \% )}{Theoretical concentration of peptide ( \% )} \times 100$$

The theoretical concentration of peptide in the composition was 0.05 wt. % in Example 1 and 0.125 wt. % in Examples 2 to 5.

TABLE 2

| | | Ex. 1 | Ex. 2 | Ex. 3 | Ex. 4 | Ex. 5 |
|---|---|---|---|---|---|---|
| Ingredient | INCI | Concentration wt. % | | | | |
| PEP-1 | Acetyl Hexapeptide-8 | 0.05 | 0.125 | 0.125 | 0.125 | 0.125 |
| Water | Aqua | 1 | 2.0 | 2.5 | 2.5 | 2.5 |
| Algapur ™ | Triolein, Tocopherol, | 58.95 | 47.875 | 17.375 | 7.375 | — |
| HSHO Algae | Helianthus Annuus | | | | | |
| Oil | Seed Oil | | | | | |
| Glucate ™ DO | Methyl Glucose | 40 | 50 | 80 | 90 | 97.375 |
| emulsifier | Dioleate | | | | | |

| | | | | | |
|---|---|---|---|---|---|
| Physical appearance | OK | KO | KO | OK | OK |

HPLC (1 week)

| | | | | | |
|---|---|---|---|---|---|
| % peptide total | 0.095 | — | — | 0.098 | 0.051 |
| % recovery at 1 week at room | 35.80 | — | — | 78.80 | 78.10 |
| temperature | | | | | |

In Examples 1, 4 and 5, it was possible to disperse the peptide in oil and the appearance was acceptable at time 0. However, more than 20% of the peptide concentration in 1 week at room temperature was lost, which is considered not acceptable. Therefore, results demonstrate that the stability of the peptide in oil was compromised.

### Examples 6 to 11

Examples 6 to 11 shown in Table 3a and 3b were prepared to study the effect of the emulsifier on the stability of the composition. Compositions which were visually stable after 24 h were stored for 3 months at different temperatures to determine the long-term stability.

The process for preparing the compositions comprised mixing to phases consists of two phases: water phase (Phase A) and oil phase (Phase B). Phase A is the solution of peptide in water. Phase B is the dispersion of Schercemol™ CO ester from Lubrizol Advanced Materials, Inc., the emulsifier (either Glucate™ SS, Glucamate™ SSE-20 or Glucate™ DO, from Lubrizol Advanced Materials, Inc.) and Hydramol™ TGL ester from Lubrizol Advanced Materials, Inc. Phase B is heated to 50-65° C. and cooled down. At room temperature, phase A is added into phase B slowly with mixing.

TABLE 3a

| | | Ex. 6 | Ex. 7 | Ex. 8 | Ex. 9 | Ex. 10 | Ex. 11 |
|---|---|---|---|---|---|---|---|
| Ingredient | INCI | Concentration in wt. % | | | | | |
| PEP-1 | Acetyl | 0.125 | 0.125 | 0.125 | 0.125 | 0.125 | 0.125 |
| | Hexapeptide-8 | | | | | | |
| Water | Aqua | 2.5 | 2.5 | 2.5 | 2.5 | 2.5 | 2.5 |
| Schercemol ™ | Cetyl | 87.375 | 86.875 | 86.375 | 85.875 | 85.375 | 84.375 |
| CO ester | Ethylhexanoate | | | | | | |
| Glucate ™ SS | Methyl Glucose | — | 0.5 | 1 | 1.5 | 2 | 3 |
| emulsifier | Sesquistearate | | | | | | |
| Glucamate ™ | PEG-20 Methyl | — | — | — | — | — | — |
| SSE-20 | Glucose | | | | | | |
| emulsifier | Sesquistearate | | | | | | |
| Glucate ™ DO | Methyl Glucose | — | — | — | — | — | — |
| emulsifier | Dioleate | | | | | | |
| Hydramol ™ | Polyglyceryl-3 | 10 | 10 | 10 | 10 | 10 | 10 |
| TGL ester | Laurate | | | | | | |

| | | | | | | |
|---|---|---|---|---|---|---|
| Physical appearance | KO | KO | OK | OK | OK | KO |

TABLE 3b

| | | Ex. 12 | Ex. 13 |
|---|---|---|---|
| Ingredient | INCI | wt. % | |
| PEP-1 | Acetyl Hexapeptide-8 | 0.125 | 0.125 |
| Water | Aqua | 2.5 | 2.5 |
| Schercemol ™ CO ester | Cetyl Ethylhexanoate | 86.375 | 86.375 |
| Glucate ™ SS | Methyl Glucose | — | — |
| emulsifier | Sesquistearate | | |
| Glucamate ™ SSE-20 | PEG-20 Methyl Glucose | 1 | — |
| emulsifier | Sesquistearate | | |
| Glucate ™ DO | Methyl Glucose Dioleate | — | 1 |
| emulsifier | | | |
| Hydramol ™ TGL ester | Polyglyceryl-3 Laurate | 10 | 10 |

| | | |
|---|---|---|
| Physical appearance | KO | KO |

Glucate™ SS emulsifier was tested at concentrations from 0 wt. % to 3 wt % based on the total weight of the composition (Examples 6 to 11). When used at 0%, 0.5% and 3% an unacceptable precipitate was observed at the bottom, while at concentrations from 1% to 2% the compositions were stable.

When the same composition was tested using Glucamate™ SSE-20 emulsifier at 1 wt. % (Example 12) or Glucate™ DO emulsifier at 1 wt. % (Example 13) an unacceptable precipitate was observed in both compositions. Samples with Glucate™, SS emulsifier at 1 wt. % (Example 8) were analyzed by HPLC and practically all the concentration of the peptide was recovered after 3 months of stability (Table 4).

TABLE 4

| | | Conc | | % |
|---|---|---|---|---|
| | | total | Normalized | concentration |
| Time | Condition | (wt. %) | data vs time 0 | variation |
| 0 h | No mixing | 0.148 | 93.6 | — |
| | Mixing | 0.158 | 100.0 | — |
| 2 weeks | 40° C. | 0.154 | 97.3 | −2.7 |
| | Room temp. | 0.152 | 96.4 | −3.6 |
| 1 month | 40° C. | 0.146 | 92.7 | −7.3 |
| | Room temp. | 0.147 | 93.1 | −6.9 |
| | −20° C. | 0.158 | 99.9 | −0.1 |
| 2 months | 40° C. | 0.141 | 89.1 | −10.9 |
| | Room temp. | 0.15 | 95.3 | −4.7 |
| 3 months | 40° C. | 0.141 | 89.4 | −10.6 |
| | 40° C. without | 0.147 | 92.9 | −7.1 |
| | opening | | | |
| | Room temp. | 0.158 | 100.2 | 0.2 |

### Examples 14-18

Different compositions as shown in Table 5 were prepared to assess the effect of the solubilizer in the oil phase in the stability of the composition.

Hydramol™ TGL ester was tested at concentrations from 5% to 15% by weight (Examples 14 to 17). When used at 5%, 8% and 15% an unacceptable precipitate was observed at the bottom, while at concentrations from 10% to 12% the composition was stable.

When the same compositions were tested using Plurol Diisostearique CG™ from Gattefosse at 10% (Example 18) an unacceptable precipitate was observed.

TABLE 5

| | | EX. 14 | EX. 15 | EX. 16 | EX. 17 | EX. 18 |
|---|---|---|---|---|---|---|
| Ingredient | INCI | wt. % | | | | |
| PEP-1 | Acetyl Hexapeptide-8 | 0.125 | 0.125 | 0.125 | 0.125 | 0.125 |
| Water | Aqua | 2.5 | 2.5 | 2.5 | 2.5 | 2.5 |
| Schercemol ™ CO | Cetyl Ethylhexanoate | 91.375 | 88.875 | 84.375 | 81.875 | 86.375 |
| ester | | | | | | |
| Glucate ™ SS | Methyl Glucose | 1 | 1 | 1 | 1 | 1 |
| emulsifier | Sesquistearate | | | | | |
| Hydramol ™ TGL | Polyglyceryl-3 | 5 | 8 | 12 | 15 | — |
| ester | Laurate | | | | | |
| Plurol | Polyglyceryl-2 | | | | | 10 |
| Diisostearique CG | Diisostearate | | | | | |

| | | | | | |
|---|---|---|---|---|---|
| Physical appearance | KO | KO | OK | KO | KO |

### Examples 19-22

Different compositions as shown in Table 6 were prepared in order to determine the influence of the amount of water in the composition.

Concentration of water ranging from 2% to 7% by weight were tested. When used at 2 wt. % (Example 19) and 7 wt. % (Example 22) an unacceptable precipitate was observed at the bottom. While at concentrations from 2.5% to 5% by weight the composition was stable.

TABLE 6

| | | Ex. 19 | Ex. 20 | Ex. 21 | Ex. 22 |
|---|---|---|---|---|---|
| Ingredient | INCI | Wt. % | | | |
| PEP-1 | Acetyl | 0.125 | 0.125 | 0.125 | 0.125 |
| | Hexapeptide-8 | | | | |
| Water | Aqua | 2 | 3 | 5 | 7 |
| Schercemol ™ | Cetyl | 96.875 | 85.875 | 83.875 | 81.875 |
| CO ester | Ethylhexanoate | | | | |
| Glucate ™ | Methyl Glucose | 1 | 1 | 1 | 1 |
| SS emulsifier | Sesquistearate | | | | |
| Hydramol ™ | Polyglyceryl-3 | 10 | 10 | 10 | 10 |
| TGL ester | Laurate | | | | |

| | | | | |
|---|---|---|---|---|
| Physical appearance | KO | OK | OK | KO |

### Examples 23-25

Different compositions were prepared to determine the relevance of the ester oil (Table 7).

Isopropyl Isostearate and Ethylhexyl Palmitate (Examples 23 and 24, respectively) were tested at 86.375 wt %. Some particles were observed at the bottom after 24h. Diisopropyl Adipate (Example 25) was tested as a solvent at 86.375% and an unacceptable precipitate was observed right after mixing.

TABLE 7

| Ingredient | INCI | Ex. 23 | Ex. 24 | Ex. 25 |
|---|---|---|---|---|
| PEP-1 | Acetyl Hexapeptide-8 | 0.125 | 0.125 | 0.125 |
| Water | Aqua | 2.5 | 2.5 | 2.5 |
| Schercemol ® 318 | Isopropyl Isostearate | 86.375 | — | — |
| Octyl palmitate | Ethylhexyl Palmitate | — | 86.375 | — |
| Schercemol ® DIA | Diisopropyl Adipate | — | — | 86.375 |
| Glucate ™ SS | Methyl Glucose | 1 | 1 | 1 |
| emulsifier | Sesquistearate | | | |
| Hydramol ™ TGL | Polyglyceryl-3 Laurate | 10 | 10 | 10 |
| ester | | | | |

| | | | |
|---|---|---|---|
| Physical appearance | OK | OK | KO |

### Examples 26-28

Different peptides with known cosmetic properties were tested using the ingredients at the optimal concentrations determined previously (Table 8).

PEP-2, PEP-8 and PEP-9 (Example 26, 27, 28, respectively) had a good compatibility and a composition with acceptable physical appearance was obtained.

TABLE 8

| Ingredient | INCI | Log P | Ex. 26 | Ex. 27 | Ex. 28 |
|---|---|---|---|---|---|
| PEP-2 | | −5.84 | 0.125 | | |
| PEP-8 | | −3.38 | | 0.125 | |
| PEP-9 | | −1.02 | | | 0.125 |
| Water | Aqua | | 2.5 | 2.5 | 2.5 |
| Schercemol ™ | Cetyl | | 86.375 | 86.375 | 86.375 |
| CO ester | Ethylhexanoate | | | | |
| Glucate ™ | Methyl Glucose | | 1 | 1 | 1 |
| SS emulsifier | Sesquistearate | | | | |
| Hydramol ™ | Polyglyceryl-3 | | 10 | 10 | 10 |
| TGL ester | Laurate | | | | |

| | | | |
|---|---|---|---|
| Physical appearance | OK | OK | OK |

### Example 29

An oil serum comprising the composition of Example 8 was prepared from the ingredients shown in Table 9.

TABLE 9

| Phase | Material Name | Mixed INCI | wt. % |
|---|---|---|---|
| A | SilSense ™ Bio 5 emollient | C9-12 ALKANE | 40.0 |
| A | MIGLYOL Coco 810, RSPO-MB | COCO-CAPRYLATE/CAPRATE | 26.6 |
| | | TRIOLEIN | |
| A | AlgaPūr ™ High Stability High | TOCOPHEROL | 20.3 |
| | Oleic (HSHO) Algae Oil | HELIANTHUS ANNUUS | |
| | | (SUNFLOWER) SEED OIL | |
| A | JOJOBA SEED OIL | SIMMONDSIA CHINENSIS | 10.0 |
| | | (JOJOBA) SEED OIL | |
| A | TOCOPHERYL ACETATE | TOCOPHERYL ACETATE | 0.5 |
| B | Example 8 | CETYL ETHYLHEXANOATE | 0.8 |
| | | POLYGLYCERYL-3 LAURATE | |
| | | WATER (AQUA) | |
| | | METHYL GLUCOSE | |
| | | SESQUISTEARATE | |
| | | ACETYL HEXAPEPTIDE-8 | |
| B | BABY ALOE VERA 54.640.1361 | FRAGRANCE (PARFUM) | 0.2 |
| B | GLUCATE ™ DO emulsifier | METHYL GLUCOSE DIOLEATE | 1.6 |
| | | TOCOPHEROL | |
| | | | 100.0 |

### Example 30

A makeup cream foundation comprising the composition of Example 8 was prepared from the ingredients shown in Table 10.

TABLE 10

| Phase | Material Name | Mixed INCI | Wt. % |
|---|---|---|---|
| A | SilSense ™ Bio 5 emollient | C9-12 ALKANE | 4.00 |
| A | MIGLYOL Coco 810, RSPO- | COCO-CAPRYLATE/CAPRATE | 7.15 |
| | MB | | |
| A | AEROSIL ® R 972 | SILICA DIMETHYL SILYLATE | 2.00 |
| A | BRB SG 516 | DIMETHICONE | 5.00 |
| | | DIMETHICONE/VINYL | |
| | | DIMETHICONE CROSSPOLYMER | |
| A | DC FZ-3196 | CAPRYLYL METHICONE | 2.00 |
| A | IMWITOR 600 | POLYGLYCERYL-3 | 2.50 |
| | | POLYRICINOLEATE | |
| A | GLUCATE ™ DO emulsifier | METHYL GLUCOSE DIOLEATE | 2.50 |
| | | TOCOPHEROL | |
| A | MATRIFUSE ™ S-1 | POLYHYDROXYSTEARIC ACID | 2.00 |
| | dispersant | NEOPENTYL GLYCOL | |
| | | DIETHYLHEXANOATE | |
| A | BENTONE GEL ISD V | ISODODECANE | 7.00 |
| | | DISTEARDIMONIUM HECTORITE | |
| | | PROPYLENE CARBONATE | |
| A | Example 8 | CETYL ETHYLHEXANOATE | 2.00 |
| | | POLYGLYCERYL-3 LAURATE | |
| | | WATER (AQUA) | |
| | | METHYL GLUCOSE | |
| | | SESQUISTEARATE | |
| | | ACETYL HEXAPEPTIDE-8 | |
| B | UNIPURE WHITE LC981 | TITANIUM DIOXIDE (CI 77891) | 11.13 |
| | HLC | HYDROGENATED LECITHIN | |
| B | UNIPURE YELLOW LC182 | IRON OXIDES (CI 77492) | 3.00 |
| | HLC | HYDROGENATED LECITHIN | |
| B | UNIPURE RED LC381 HLC | IRON OXIDES (CI 77491) | 0.52 |
| | | HYDROGENATED LECITHIN | |
| B | UNIPURE BLACK LC989 | IRON OXIDES (CI 77499) | 0.35 |
| | HLC | HYDROGENATED LECITHIN | |
| C | WATER | WATER (AQUA) | 41.75 |
| C | ZEMEA PROPANEDIOL | PROPANEDIOL | 5.00 |
| C | DERMOSOFT GMCY, | GLYCERYL CAPRYLATE | 1.00 |
| | RSPO-MB | | |
| C | Magnesium Sulfate, 7H₂O | MAGNESIUM SULFATE | 0.75 |
| C | SODIUM BENZOATE | SODIUM BENZOATE | 0.30 |
| C | BABY ALOE VERA | FRAGRANCE (PARFUM) | 0.05 |
| | 54.640.1361 | | |
| | | | 100.00 |

### Example 31

A facial serum stick comprising the composition of Example 8 was prepared from the ingredients shown in Table 11.

TABLE 11

| Phase | Material Name | Mixed INCI | Wt. % |
|---|---|---|---|
| A | Caprylic/Capric Triglyceride | CAPRYLIC/CAPRIC TRIGLYCERIDE | 27.45 |
| A | Schercemol ™ NGDO ester | NEOPENTYL GLYCOL | 25.00 |
| | | DIETHYLHEXANOATE | |
| A | Schercemol ™ 1818 ester | ISOSTEARYL ISOSTEARATE | 10.00 |
| A | Oilkemia ™ 5S polymer | CAPRYLIC/CAPRIC TRIGLYCERIDE | 1.00 |
| | | POLYURETHANE-79 | |
| A | MSS-500 | SILICA | 5.00 |
| A | Floraesters 60 | JOJOBA ESTER | 1.50 |
| A | KAHLWAX 2442 | COPERNICIA CERIFERA | 4.00 |
| | | (CARNAUBA) WAX | |
| A | Candelilla Wax NF | EUPHORBIA CERIFERA | 7.00 |
| | | (CANDELILLA) WAX | |
| A | Kahlwax 2811 | OYZA SATIVA (RICE) BRAN WAX | 5.00 |
| A | Glucate ™ DO emulsifier | METHYL GLUCOSE DIOLEATE | 1.00 |
| B | Algapür ™ HSHO Algae Oil | TRIOLEIN | 5.00 |
| B | KoboBlur ™ 100 Natural | SILICA | 5.00 |
| | | CELLULOSE | |
| | | MICA | |
| | | BARIUM SULFATE | |
| | | TITANIUM DIOXIDE | |
| | | JOJOBA ESTERS | |
| B | Lipochroman ™ molecule | DIMETHYLMETHOXY CHROMANOL | 0.05 |
| B | Example 8 | CETYL ETHYLHEXANOATE | 2.00 |
| | | POLYGLYCERYL-3 LAURATE | |
| | | WATER (AQUA) | |
| | | METHYL GLUCOSE | |
| | | SESQUISTEARATE | |
| | | ACETYL HEXAPEPTIDE-8 | |
| B | Lexgard Natural MB ™ | GLYCERYL CAPRYLATE | 1.00 |
| | | GLYCERYL UNDECYLENATE | |
| | | | 100.00 |

### Example 32

A cream-to-powder mouse was prepared from the ingredients shown in Table 12:

TABLE 12

| Phase | Material Name | Mixed INCI | Wt. % |
|---|---|---|---|
| A | Kahlwax 2811 Rice Bran | ORYZA SATIVA BRAN WAX | 4.00 |
| | Wax | | |
| A | ARLACEL 165 ™ | GLYCERYL STEARATE | 2.00 |
| | (LIPOMULSE) | PEG-100 STEARATE | |
| A | CETEARYL ALCOHOL | CETEARYL ALCOHOL | 5.00 |
| | (LANETTE O OR) - COG | | |
| A | GLYCERYL STEARATE | GLYCERYL STEARATE | 6.00 |
| A | Natura Velvet Apricot | PRUNUS ARMENIACA KERNEL OIL | 10.00 |
| | | HYDROGENATED VEGETABLE OIL | |
| | | BUTYROSPERMUM PARKII BUTTER | |
| | | TOCOPHEROL | |
| B | BENTONE GEL ISD V | ISODODECANE | 20.00 |
| | (EP080) | DISTEARDIMONIUM HECTORITE | |
| | | PROPYLENE CARBONATE | |
| B | SCHERCEMOL ™ 1818 | ISOSTEARYL ISOSTEARATE | 26.20 |
| B | Vivapur CS 9 FM ™ | MICROCRYSTALLINE CELLULOSE | 12.00 |
| C | COLORONA KARAT GOLD | MICA | 1.00 |
| | MP-24 (CO105) | CI 77891 | |
| | | CI 77491 | |
| C | COLORONA BRONZE FINE | MICA | 3.00 |
| | (CO103) | CI 77491 | |
| D | Silsense ™ Bio 5 Emollient | C9-12 ALKANE | 10.00 |
| D | Example 8 | CETYL ETHYLHEXANOATE | 0.80 |
| | | POLYGLYCERYL-3 LAURATE | |
| | | AQUA | |
| | | METHYL GLUCOSE | |
| | | SESQUISTEARATE | |
| | | ACETYL HEXAPEPTIDE-8 | |
| | | | 100.00 |

### Example 33

A biphasic serum was prepared from the ingredients shown in Table 13.

TABLE 13

| Phase | Material Name | EU INCI | % wt. |
|---|---|---|---|
| A | WATER | WATER (AQUA) | 22.18 |
| A | Oxylance ™ advanced | GLYCERIN | 5.0 |
| | botanical ingredient | WATER (AQUA) | |
| | | LIGUSTRUM LUCIDUM SEED | |
| | | EXTRACT | |
| A | Xpozuki ™ biotech ingredient | WATER (AQUA) | 2.0 |
| | | LEUCONOSTOC/RADISH ROOT | |
| | | FERMENT FILTRATE | |
| | | PHASEOLUS ANGULARIS SEED | |
| | | EXTRACT | |
| | | CITRIC ACID | |
| A | GLYCERIN | GLYCERIN | 15.0 |
| A | DERMOSOFT HEXIOL | 1,2-HEXANEDIOL | 0.5 |
| A | SymSave H ™ | HYDROXYACETOPHENONE | 0.5 |
| A | STEVISSE ™ advanced | GLYCERIN | 2.0 |
| | botanical ingredient | WATER (AQUA) | |
| | | STEVIA REBAUDIANA LEAF/STEM | |
| | | EXTRACT | |
| A | Sodium Chloride | SODIUM CHLORIDE | 2.0 |
| B | Schercemol LL ester | LAURYL LACTATE | 50.0 |
| B | Example 8 | CETYL ETHYLHEXANOATE | 0.8 |
| | | POLYGLYCERYL-3 LAURATE | |
| | | AQUA | |
| | | METHYL GLUCOSE | |
| | | SESQUISTEARATE | |
| | | ACETYL HEXAPEPTIDE-8 | |
| B | BABY ALOE VERA | PARFUM | 0.0 |
| | | | 100.0 |

The invention can be further described by way of the numbered clauses below:
1. A composition comprising:
i) from 2 to 5 wt. % of an aqueous solution comprising a hydrophilic active ingredient;
ii) from 1 to 2 wt. % of methyl glucose sesquistearate;
iii) from 10 to 12 wt. % of a polyglyceryl-3 ester of a fatty acid;
iv) at least 83 wt. % of an ester oil selected from the group consisting of cetyl ethylhexanoate, isopropyl isostearate and ethylhexyl palmitate; and
   wherein all the weight percentages are based on the total weight of the composition; and,
wherein the sum of i), ii), iii) and iv) does not exceed 100 wt. %.
2. A composition according to clause 1 wherein the polyglycerol-3 ester of fatty acid is triglycerol laurate.
3. A composition according to clauses 1 or 2 wherein the ester oil is cetyl ethylhexanoate.
4. A composition according to any one of the previous clauses wherein the active ingredient is a cosmetic active ingredient.
5. A composition according to any one of the previous clauses wherein the active ingredient is a peptide, a botanical extract or an ingredient obtained from a biotechnological process.
6. A composition according to clause 5 wherein the peptide is selected from the group consisting of tripeptides, tetrapeptides, pentapeptides, hexapeptides, heptapeptides, octapeptides and nonapeptides.
7. A composition according to clause 6, wherein the peptide is one or more peptides selected from the group consisting of:

(SEQ ID. 1)
R₁-Glu-Glu-Met-Gln-Arg-Arg-R₂;
(SEQ ID. 2)
R₁-Glu-Glu-Met-Gln-Arg-Arg-Ala-Asp-R₂;
(SEQ ID. 3)
R₁-Glu-Glu-Met-Gln-Arg-Arg-Ala-R₂;
(SEQ ID. 4)
R₁-Arg-Arg-GIn-Met-Glu-Glu-R₂;
(SEQ ID. 5)
R₁-Arg-Arg-D-Gln-Met-Glu-Glu-R₂;
(SEQ ID. 6)
R₁-Arg-Arg-Gln-D-Met-Glu-Glu-R₂;
(SEQ ID. 7)
R₁-Arg-Arg-D-Gln-D-Met-Glu-Glu-R₂;
(SEQ. ID. 8)
R₁-Phe-Trp-Met-Lys-Arg-Lys-Arg-Val-Pro-R₂;
(SEQ. ID. 9)
R₁-Tyr-D-Ala-Gly-Phe-Leu-R₂;
and,
(SEQ. ID. 10)
R₁-Leu-Pro-Val-Thr-R₂.

wherein R₁ is selected from the group consisting of H and R₅—CO—, wherein R₅ is selected from the group consisting of C₁-C₁₈ alkyl, C₂-C₂₄ alkenyl, C₃-C₂₄ cycloalkyl; and R₂ is —NR₃R₄ or —OR₃ wherein R₃ and R₄ are independently selected from the group consisting of H and C₁-C₁₆ alkyl.
8. A composition according to any one of clauses 6 to 7 wherein the peptide is one or more peptides selected from the group consisting of:

Ac-Glu-Glu-Met-Gln-Arg-Arg-NH₂ (PEP-1);
Ac-Glu-Glu-Met-Gln-Arg-Arg-Ala-Asp-NH₂ (PEP-2);
Ac-Glu-Glu-Met-Gln-Arg-Arg-Ala-NH₂ (PEP-3);
Ac-Arg-Arg-GIn-Met-Glu-Glu-NH₂ (PEP-4);
Ac-Arg-Arg-D-Gln-Met-Glu-Glu-NH₂ (PEP-5);
Ac-Arg-Arg-Gln-D-Met-Glu-Glu-NH₂ (PEP-6);
Ac-Arg-Arg-D-Gln-D-Met-Glu-Glu-NH₂ (PEP-7);
H-Phe-Trp-Met-Lys-Arg-Lys-Arg-Val-Pro-NH₂
(PEP-8);
H-Tyr-D-Ala-Gly-Phe-Leu-OH (PEP-9);
and,
H-Leu-Pro-Val-Thr-OH (PEP-10).

9. A composition according to clause 8 wherein the peptide is Ac-Glu-Glu-Met-Gln-Arg-Arg-NH₂, Ac-Arg-Arg-Gln-D-Met-Glu-Glu-NH₂ or H-Tyr-D-Ala-Gly-Phe-Leu-OH.
10. A composition according to clause 9, wherein the peptide is Ac-Glu-GIu-Met-Gln-Arg-Arg-NH₂.
11. A composition according to any one of the previous clauses wherein the amount of aqueous solution i) is from 2 to 3 wt. % based on the total weight of the composition.
12. A composition according to any one of the previous clauses wherein the amount of aqueous solution i) is from 2 to 2.5 wt. % based on the total weight of the composition.
13. A composition according to any one of the previous clauses wherein the amount of methyl glucose sesquistearate is from 1 to 1.5 wt. % based on the total weight of the composition.
14. A composition according to any one of previous clauses wherein the active ingredient is in an amount from 0.01 to 0.5 wt. %, or from 0.05 to 0.25 wt. % or from 0.1 to 0.2 wt. % based on the total weight of the composition.
15. A method for dispersing a hydrophilic active ingredient in oil comprising the steps of:
   (i) Dissolving a hydrophilic active ingredient in an aqueous solution; and
   (ii) Mixing the aqueous solution obtained in step i) with an oil system comprising a combination of methyl glucose sesquistearate, a polyglyceryl-3 ester of a fatty acid and an ester oil selected from the group consisting of cetyl ethylhexanoate, isopropyl isostearate and ethylhexyl palmitate.
16. A method according to clause 15, wherein the oil phase is heated at 50-65° C. before mixing.
17. A method according to clauses 15 or 16, wherein the active ingredient is a peptide.
18. A method according to any one of clauses 15 to 17 wherein the polyglyceryl-3 ester of a fatty acid is triglycerol laurate.
19. A method according to any one of clauses 15 to 18 wherein the ester oil is cetyl ethylhexanoate.
20. A method according to any one of clauses 15 to 19, wherein the active ingredient is dissolved in water.
21. A cosmetic or dermopharmaceutical formulation which comprises the composition according to any one of clauses 1 to 14, together with at least one cosmetically or dermopharmaceutically acceptable excipient or adjuvant.
22. A cosmetic or dermopharmaceutical formulation according to clause 21 wherein the concentration of the composition according to clauses 1 to 14 is in an amount from 0.001 to 10 wt. %, preferably from 0.004 to 5 wt. % based on the total weight of the cosmetic or dermopharmaceutical formulation.
23. A cosmetic or dermopharmaceutical formulation according to clauses 21 or 22, wherein said formulation is in the form of a creams, multiple emulsion, foundation, solution, anhydrous formulation, dispersion, oil, milk, balsam, lotion, gel, cream gels, liniment, conditioner, serum, stick, ointment, mousse, pomade, powder, bar, pencil, spray, aerosol or mask.
24. A cosmetic or dermopharmaceutical formulation according to any one of clauses 21 and 22, wherein said formulation is incorporated into a fabric or a device.
25. A method of cosmetic, non-therapeutic treatment and/or care of the skin, hair, nails and/or mucous membranes of a subject comprising topically administering a cosmetically effective amount of a composition according to any one of clauses 1 to 14 or a cosmetic formulation according to any one of clauses 21 to 24.
26. A method according to clause 25, wherein the cosmetic, non-therapeutic treatment and/or care is: the treatment and/or prevention of skin aging; the reduction and/or prevention of skin wrinkles.
27. Use of a composition according to any one of clauses 1 to 14, for the cosmetic, non-therapeutic treatment and/or care of the skin, hair, nails and/or mucous membranes of a subject.

A sequence listing in XML format is submitted herewith. The name of the file is 4769-01-WO Sequence listing 2021.02.01. The file was created Aug. 1, 2025. The file size is 11,090 bytes. The sequence listing adds no new matter.

1. A composition comprising:
i) from 2 to 5 wt. % of an aqueous solution comprising a hydrophilic active ingredient;
ii) from 1 to 2 wt. % of methyl glucose sesquistearate;
iii) from 10 to 12 wt. % of a polyglyceryl-3 ester of a fatty acid;
iv) at least 83 wt. % of an ester oil selected from the group consisting of cetyl ethylhexanoate, isopropyl isostearate and ethylhexyl palmitate; and
wherein all the weight percentages are based on the total weight of the composition;
and, wherein the sum of i), ii), iii) and iv) does not except 100 wt. %.

2. The composition according to claim 1 wherein the polyglyceryl-3 ester of the fatty acid is triglycerol laurate.

3. The composition according to claim 1, wherein the ester oil is cetyl ethylhexanoate.

4. The composition according to claim 1, wherein the active ingredient is a cosmetic active ingredient.

5. The composition according to claim 1, wherein the active ingredient is a peptide.

6. The composition according to claim 5, wherein the peptide is one or more peptides selected from the group consisting of:
(SEQ ID. 1)
R₁-Glu-Glu-Met-Gln-Arg-Arg-R₂;
(SEQ ID. 2)
R₁-Glu-Glu-Met-Gln-Arg-Arg-Ala-Asp-R₂;
(SEQ ID. 3)
R₁-Glu-Glu-Met-Gln-Arg-Arg-Ala-R₂;
(SEQ ID. 4)
R₁-Arg-Arg-Gln-Met-Glu-Glu-R₂;
(SEQ ID. 5)
R₁-Arg-Arg-D-Gln-Met-Glu-Glu-R₂;
(SEQ ID. 6)
R₁-Arg-Arg-Gln-D-Met-Glu-Glu-R₂;
(SEQ ID. 7)
R₁-Arg-Arg-D-Gln-D-Met-Glu-Glu-R₂;
(SEQ. ID. 8)
R₁-Phe-Trp-Met-Lys-Arg-Lys-Arg-Val-Pro-R₂;
(SEQ. ID. 9)
R₁-Tyr-D-Ala-Gly-Phe-Leu-R₂;
and,
(SEQ. ID. 10)
R₁-Leu-Pro-Val-Thr-R₂;
wherein R₁ is selected from the group consisting of H and R₅—CO—, wherein R₅ is selected from the group consisting of C₁-C₁₈ alkyl, C₂-C₂₄ alkenyl, C₃-C₂₄ cycloalkyl; and R₂ is —NR₃R₄ or —OR₃ wherein R₃ and R₄ are independently selected from the group consisting of H and C₁-C₁₆ alkyl.

7. The composition according to claim 5, wherein the peptides is one or more peptides selected from the group consisting of:
Ac-Glu-Glu-Met-Gln-Arg-Arg-NH₂ (PEP-1);
Ac-Glu-Glu-Met-Gln-Arg-Arg-Ala-Asp-NH₂ (PEP-2);
Ac-Glu-Glu-Met-Gln-Arg-Arg-Ala-NH₂ (PEP-3);
Ac-Arg-Arg-Gln-Met-Glu-Glu-NH₂ (PEP-4);
Ac-Arg-Arg-D-Gln-Met-Glu-Glu-NH₂ (PEP-5);
Ac-Arg-Arg-Gln-D-Met-Glu-Glu-NH₂ (PEP-6);
Ac-Arg-Arg-D-Gln-D-Met-Glu-Glu-NH₂ (PEP-7);
H-Phe-Trp-Met-Lys-Arg-Lys-Arg-Val-Pro-NH₂
(PEP-8);
H-Tyr-D-Ala-Gly-Phe-Leu-OH (PEP-9);
and,
H-Leu-Pro-Val-Thr-OH (PEP-10).

8. The composition according to claim 1, wherein the amount of aqueous solution i) is from 2 to 3 wt. % based on the total weight of the composition.

9. The composition according to claim 1, wherein the amount of methyl glucose sesquistearate is from 1 to 1.5 wt. % based on the total weight of the composition.

10. The composition according to claim 1, wherein the active ingredient is in an amount from 0.01 to 0.5 wt. %, based on the total weight of the composition.

11. A method for dispersing a hydrophilic active ingredient in oil comprising the steps of:
(i) dissolving a hydrophilic active ingredient in an aqueous solution; and
(ii) mixing the aqueous solution obtained in step i) with an oil system comprising amethyl glucose sesquistearate, a polyglyceryl-3 ester of a fatty acid and an ester oil selected from the group consisting of cetyl ethylhexanoate, isopropyl isostearate and ethylhexyl palmitate.

12. A cosmetic or dermopharmaceutical formulation which comprises the composition according to claim 1, and at least one cosmetically or dermopharmaceutically acceptable excipient or adjuvant.

13. The cosmetic or dermopharmaceutical formulation according to claim 12 wherein the composition according to claim 1 is in an amount from 0.001 to 10 wt. %, based on the total weight of the cosmetic or dermopharmaceutical formulation.

14. A method of cosmetic, non-therapeutic treatment and/or care of the skin, hair, nails and/or mucous membranes of a subject comprising topically administering to said skin, hair, nails and/or mucous membranes of the subject a cosmetically effective amount of the cosmetic formulation according to claim 12.

15. (canceled)


SEQUENCE LISTING (10 sequences)

SEQ ID NO: 1 - protein, 6 aa, synthetic construct
EEMQRR

SEQ ID NO: 2 - protein, 8 aa, synthetic construct
EEMQRRAD

SEQ ID NO: 3 - protein, 7 aa, synthetic construct
EEMQRRA

SEQ ID NO: 4 - protein, 6 aa, synthetic construct
RRQMEE

SEQ ID NO: 5 - protein, 6 aa, synthetic construct
  SITE 3: note = D-Gln
RRQMEE

SEQ ID NO: 6 - protein, 6 aa, synthetic construct
  SITE 4: note = D-Met
RRQMEE

SEQ ID NO: 7 - protein, 6 aa, synthetic construct
  SITE 3: note = D-Gln
  SITE 4: note = D-Met
RRQMEE

SEQ ID NO: 8 - protein, 9 aa, synthetic construct
FWMKRKRVP

SEQ ID NO: 9 - protein, 5 aa, synthetic construct
  SITE 2: note = D-Ala
YAGFL

SEQ ID NO: 10 - protein, 4 aa, synthetic construct
LPVT